(19) United States
(12) Patent Application Publication
(10) Pub. No.: US 2024/0269192 A1
(43) Pub. Date: Aug. 15, 2024

(54) FIBROBLAST BASED THERAPEUTICS OF AMYOTROPHIC LATERAL SCLEROSIS

(71) Applicant: SPINALCYTE LLC, Webster, TX (US)

(72) Inventors: Thomas ICHIM, San Diego, CA (US); Pete O'HEERON, Houston, TX (US)

(73) Assignee: SPINALCYTE LLC, Webster, TX (US)

(21) Appl. No.: 18/570,530

(22) PCT Filed: Jun. 17, 2022
(86) PCT No.: PCT/US2022/034062

§ 371 Date: Dec. 14, 2023

Related U.S. Application Data
(60) Provisional application No. 63/211,989, filed on Jun. 17, 2021.

(51) Int. Cl. A61K 35/33; A61K 38/20; A61K 45/06

(52) U.S. Cl. CPC A61K 35/33; A61K 38/2013; A61K 45/06

(57) ABSTRACT

The present disclosure concerns methods and compositions for treating or preventing or reducing the risk of having Amyotrophic Lateral Sclerosis (ALS) in an individual. In particular embodiments, there are methods and compositions related to administering to the individual a therapeutically effective amount of a population of fibroblasts, fibroblast exosomes, IL-2, or a combination thereof.

### CROSS-REFERENCE TO RELATED APPLICATIONS

This application claims priority to U.S. Provisional Patent Application Ser. No. 63/211,989, filed Jun. 17, 2021, which is incorporated by reference herein in its entirety.

### TECHNICAL FIELD

Embodiments of the disclosure encompass at least the fields of cell biology, molecular biology, physiology, and medicine.

### BACKGROUND

Amyotrophic lateral sclerosis (ALS) is a progressive neurodegenerative condition causing muscular atrophy and death within 3-5 years after its onset [1]. In the majority of patients (90%) the cause of ALS is idiopathic; however, in about 10% of the patients a familial form of the disease is presented [2]. Specific muscular degeneration is exclusive to motor neurons and begins focally and spreads, leading to weakness of limb, respiratory, and bulbar muscles. Immediately preceding death, there is a near total loss of limb and respiratory function, as well as a loss of the ability to chew, swallow, and speak.

In the United States, ALS is defined as an “orphan disease,” with approximately 2 per 100,000 new cases per year and a prevalence of about 5 per 100,000 total cases each year [3]. In the United States [4] and Europe [5], ALS is diagnosed in about 1 in 500 to 1 in 1,000 adult deaths, implying that 500,000 people in the United States will develop this disease in their lifetimes. About 10% of ALS cases are inherited, usually as dominant traits [6]. Both familial ALS (fALS) and sporadic ALS (sALS) can develop concurrently with frontotemporal lobar dementia (FTLD). By contrast, with the dementia of Alzheimer's disease (AD), in which the cardinal finding is memory loss, FTLD is characterized by behavioral changes and progressive aphasia, sometimes accompanied by movement disorders. While AD involves prominent pathology in the hippocampus, the essential finding in FTLD is, as the name suggests, early atrophy of the frontal and temporal lobes. Four recurring themes have emerged from the pathological analysis of autopsied cases with sALS, fALS, or ALS-FTLD with diverse genetic causes. First, the motor neuron death usually entails deposition of aggregated proteins, often ubiquitinated and predominantly cytoplasmic. Second, in ALS, the levels and functions of RNA and RNA-binding proteins are abnormal. Aggregates of protein and RNA are detected both in motor neurons and non-neuronal cells, such as astrocytes and microglia. Third, most cases entail some disturbance of neuronal cytoskeletal architecture and function. Additionally, in almost all cases, motor neuron death is influenced by non-neuronal cells, including oligodendroglia and cells involved in neuroinflammation (e.g., astroglia and microglia).

The gene most commonly associated with ALS is the C9ORF72 gene having repeat expansions of a non-coding GGGGCC hexanucleotide repeat [7], which affects approximately 40% of cases of familial ALS [8], and in some cases it is associated with frontotemporal dementia [9]. The abnormal repeats in the C9ORF72 gene mechanistically contribute to the biology of disease progression. An interesting study demonstrated some significant possible mechanisms using an elegant in vitro model. Specifically, induced pluripotent stem cell (iPSC)-differentiated neurons from C9ORF72 ALS patients revealed disease-specific a) intranuclear GGGGCCexp RNA foci, (b)) dysregulated gene expression, (c) sequestration of GGGGCCexp RNA binding protein ADARB2, and (d) susceptibility to excitotoxicity. These pathological and pathogenic characteristics were confirmed in ALS patients' brains and were abrogated with antisense oligonucleotide mediated inhibition of the C9ORF72 transcript or repeat expansion despite the presence of repeat-associated non-ATG translation (RAN) products. According to the authors, their data indicate a toxic RNA gain-of-function mechanism as a cause of C9ORF72 ALS and provide candidate antisense therapeutics and candidate human pharmacodynamic markers for therapy [10]. The importance of C9ORF72 repeats in neurodegeneration is supported by studies that demonstrate that these repeats are found not only in ALS patients but also in patients with Alzheimer's disease [11, 12], Parkinson's disease [13], and other dementias [14]. The Cu/Zn-superoxide dismutasel (SOD1), is also a major genetic association with ALS pathogenesis. Additional, less common genes associated with ALS include: TAR DNA-binding protein 43 (TARDBP), fused in sarcoma (FUS) and other less frequent mutations.

Despite the significant advances in knowledge of ALS pathology, presently the only available treatment is riluzole, which extends the survival time by only three months, with no improvement in the quality of life. Therefore, it is imperative to search for new alternatives to treat ALS, and the present disclosure provides such a solution.

### BRIEF SUMMARY

The present invention is directed to a system, methods, and compositions that are directed to reducing and/or reversing one or more symptoms of amyotrophic lateral sclerosis (ALS) in a mammal. In one embodiment, administration of fibroblasts and/or modified fibroblasts and/or fibroblast exosomes is performed, such as in order to induce immunological and/or regenerative alterations resulting in slowing down and/or reversing motorneuron degeneration associated with ALS. In one embodiment, fibroblasts and/or modified fibroblasts and/or fibroblast exosomes are utilized to generate immune modulatory cells that inhibit neural inflammation and allow for stimulation of regenerative processes. In some embodiments, fibroblasts and/or modified fibroblasts and/or fibroblast exosomes are utilized as therapeutic adjuvants.

In specific embodiments, there are methods of treating or preventing or reducing the risk of having Amyotrophic Lateral Sclerosis (ALS) in an individual, comprising administering to the individual a therapeutically effective amount of a population of fibroblasts, fibroblast exosomes, modified fibroblasts, IL-2, or a combination thereof. In some embodiments, the method further comprising administering to the individual an effective amount of rapamycin, N-acetylcysteine, anti-CD3 antibodies, or a combination thereof. The fibroblasts may be allogeneic to the individual or may be autologous or xenogeneic to the individual. In certain cases, the fibroblasts are mitotically active prior to administration into a recipient in need of treatment.

The fibroblasts may come from any source and may be isolated from a tissue selected from the group consisting of: a) skin; b) bone marrow; c) blood; d) mobilized peripheral blood; e) gingiva; f) tonsil; g) placenta; h) Wharton's Jelly; i) hair follicle; j) fallopian tube; k) liver; l) deciduous tooth; m) vas deferens; n) endometrial; o) menstrual blood; p) omentum; and q) a combination thereof.

In specific cases, the ALS in the individual is associated with an elevation of inflammatory cytokines as compared to an age-matched healthy control, and the inflammatory cytokine may be IL-1, IL-2, IL-6, IL-9, IL-11, IL-12, IL-15, IL-16, IL-17, IL-18, IL-20, IL-21, IL-22, IL-23, IL-27, IL-33, HMGB-1, TNF-alpha, TNF-beta, IFN-alpha, IFN-beta, IFN-gamma. In certain embodiments, the fibroblasts are selected for expression of CD73, CD70. CD105, CD16. CD55. CD37, interleukin-10 receptor, and/or interferon gamma receptor.

The fibroblasts may be selected for expression of CD73, subsequently treated with interferon gamma, and allowed to multiply for at least one cell division prior to administration. The fibroblasts and/or modified fibroblasts may be administered in a manner capable of stimulating generation of T regulatory cells. The T regulatory cells may express FoxP3, may comprise membrane bound TGF-beta, may suppress the ability of T cells to proliferate in response to a mitogen, and/or may suppress the ability of immature dendritic cells to mature into differentiated dendritic cells. In specific cases, the dendritic cell maturation is associated with upregulation of expression of one or more markers selected from the group consisting of: a) HLA-II; b) CD40; c) CD80; d) CD86; and e) a combination thereof. The dendritic cell maturation may be associated with enhanced ability to activate proliferation of allogeneic T cells. The dendritic cell maturation may be associated with enhanced ability to induce production of interferon gamma from allogeneic T cells. In specific embodiments, the T regulatory cells are activated by exposure to CD3, CD28. interleukin-10 and/or by administration of immature dendritic cells, which may express PD-1L. The immature dendritic cells may be kept in an immature state by culture in low dose GM-CSF, human chorionic gonadotropin, hypoxia, and/or inhibition of NF-kappa b activity. Inhibition of NF-kappa B activity may be achieved by administration of an antisense molecule targeting NF-kappa B or molecules in the NF-kappa B pathway, by administration of a molecule capable of triggering RNA interference targeting NF-kappa B or molecules in the NF-kappa B pathway, by gene editing means targeting NF-kappa B or molecules in the NF-kappa B pathway, by administration of decoy oligonucleotides capable of blocking NF-kappa B or molecules in the NF-kappa B pathway, and/or by administration of a small molecule blocker of NF-kappa B activity. The small molecule blocker of NF-kappa B activity may be selected from the group consisting of: Calagualine (fern derivative), Conophylline (Ervatamia microphylla), Evodiamine (Evodiae fructus component), Geldanamycin, Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, and/or Saline (low Na+ istonic). In certain embodiments, the T regulatory cells are activated by incubation with mesenchymal stem cell exosomes and may begenerated in vivo by exposure of T cells to an activator of interleukin-2 receptor is capable of inducing proliferation and/or activation of CD4 CD25 T cells.

In some embodiments of the methods, the interleukin-2 receptor is activated by administration of the IL-2. The IL-2 may be administered every day at concentrations of 0.3×10⁶ to 3.0×10⁶ IU IL-2 per square meter of body surface area for 1-16 weeks, in some cases.

In particular embodiments, any method may further comprise administering one or more immune modulatory compounds, such as oxytocin, prolactin, IL-10, IL-35, CD3 inhibitor, or a combination thereof. The CD3 inhibitor may be an anti-CD3 antibody, such as Teplizumab. In particular embodiments, the individual has a familial form of ALS or has an idiopathic form of ALS. The individual may have one or more mutations in the C9ORF72 gene. In any method, there may further comprise administering riluzole to the individual.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### DETAILED DESCRIPTION

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

The term “isolated” as used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, such as that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides that are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

As used herein, “prevent,” and similar words such as “prevented,” “preventing” etc., indicate an approach for preventing, inhibiting, or reducing the likelihood of the occurrence or recurrence of, a disease or condition, e.g., ALS. It also refers to delaying the onset or recurrence of a disease or condition or delaying the occurrence or recurrence of the symptoms of a disease or condition. As used herein, “prevention” and similar words also include reducing the intensity, effect, symptoms and/or burden of a disease or condition prior to onset or recurrence of the disease or condition.

The term “subject,” as used herein, generally refers to an individual having ALS or is suspected of having or is at risk for having over the general population. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having ALS or known to have it. The subject may be undergoing or having undergone treatment. The subject may be asymptomatic. The subject may be healthy individuals but that are desirous of prevention of ALS. The term “individual” may be used interchangeably, in at least some cases. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles. It is not intended that the term connote a need for medical treatment, therefore, an individual may voluntarily or involuntarily be part of experimentation whether clinical or in support of basic science studies.

As used herein “treatment” or “treating,” includes any beneficial or desirable effect on the symptoms or pathology of a disease or pathological condition, and may include even minimal reductions in one or more measurable markers of the disease or condition being treated, e.g., ALS. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, unless explicitly stated otherwise or clearly implied otherwise the terms ‘therapeutically effective dose,’ ‘therapeutically effective amounts,’ “effective amount, and the like, refers to a portion of a compound that has a net positive effect on the health and well-being of a human or other animal. Therapeutic effects may include an improvement in longevity, quality of life, reduction in the number and/or severity of one or more symptoms, and the like; these effects also may also include a reduced susceptibility to developing disease or deteriorating health or wellbeing. The effects may be immediate realized after a single dose and/or treatment or they may be cumulative realized after a series of doses and/or treatments.

As used herein, unless explicitly stated otherwise or clearly implied otherwise the term ‘about’ refers to a range of values plus or minus 10 percent, e.g. about 1.0 encompasses values from 0.9 to 1.1.

Embodiments of the disclosure provide means of treating or preventing or reducing the risk of having ALS through administration of fibroblasts and/or modified fibroblasts and/or fibroblast exosomes and/or IL-2. In specific embodiments, the disclosure encompasses the utilization of fibroblasts and/or modified fibroblasts to induce T regulatory cells (Treg) that reduce neural inflammation and allow for regenerative processes to occur.

In certain embodiments, the disclosure provides for the use of fibroblasts and/or modified fibroblasts and/or fibroblast exosomes and/or IL-2 to prevent, inhibit, delay the onset of, slow the progression of, or reverse ALS. In some embodiments of the disclosure, stimulation of T regulatory cells by fibroblasts in vivo is accomplished in order to reduce inflammation and stimulate regeneration in ALS patients. In some embodiments, methods include the administration of Aldesleukin (Proleukin, Novartis), which is a commercially available IL-2 licensed for the treatment of metastatic renal cell carcinoma in the UK. It is produced by recombinant DNA technology using an Escherichia coli strain, which contains a genetically engineered modification of the human IL-2 gene, and is administered either intravenously or subcutaneously (SC) at doses capable of selectively expanding T regulatory cells. This may be performed with and/or without fibroblasts and/or modified fibroblasts and/or fibroblast exosomes.

The IL-2 in any form may be administered at any suitable dose and by any suitable administration. The IL-2 may be administered every day at concentrations of 0.3×10⁶ to 3.0×10⁶ IU IL-2 per square meter of body surface area, and any derivable range therein, and the administration may be for 1-16 weeks, in at least some cases. In specific cases, following short intravenous infusion, its pharmacokinetic profile is typified by high plasma concentrations, rapid distribution into the extravascular space, and a rapid renal clearance. The recommended doses for continuous infusion and subcutaneous injection may be repeated cycles of 18×106 IU per m2 per 24 hours for 5 days and repeated doses of 18×106 IU, respectively. Peak plasma levels are reached in 2-6 hours after SC administration, with bioavailability of IL-2 (including aldesleukin) ranging between 31% and 47%. The process of absorption and elimination of subcutaneous IL-2 (including aldesleukin) is described by a one-compartment model, with a 45 min absorption half-life and an elimination half-life of 3-5 hours [15]. Natural IL-2 was first identified in 1976 as a growth factor for T lymphocytes. It is produced by human cluster designation (CD) 4+ and some CD8+ T-cells and is synthesized mainly by activated T-cells, in particular CD4+ helper T cells. It stimulates the proliferation and differentiation of T cells, induces the generation of cytotoxic T lymphocytes (CTLs) and the differentiation of peripheral blood lymphocytes to cytotoxic cells and lymphokine-activated killer (LAK) cells, promotes cytokine and cytolytic molecule expression by T cells, facilitates the proliferation and differentiation of B-cells and the synthesis of immunoglobulin by B-cells, and stimulates the generation, proliferation and activation of natural killer (NK) cells. IL-2 is known to play a central role in the generation of immune responses. In cancer clinical trials, high-dose recombinant IL-2 (e.g., IV bolus dose of 600,000 international units (IU)/kg every 8 hours for up to 14 doses) demonstrated antitumor activity in metastatic renal cell carcinoma (RCC) and metastatic melanoma. Accordingly, such high-dose IL-2 was approved for the treatment of metastatic RCC in Europe in 1989 and in the US in 1992. In 1998, approval was obtained to treat patients with metastatic melanoma. Recombinant human IL-2 (Aldesleukin) (Proleukin®-Novartis Inc. & Prometheus Labs Inc.) is currently approved by the United States Food and Drug Administration (US FDA). However, IL-2 has a dual function in the immune response in that it not only mediates expansion and activity of effector cells, but also is crucially involved in maintaining peripheral immune tolerance. A major mechanism underlying peripheral self-tolerance is IL-2 induced activation-induced cell death (AICD) in T cells. AICD is a process by which fully activated T cells undergo programmed cell death through engagement of cell surface-expressed death receptors such as CD95 (also known as Fas) or the TNF receptor. When antigen-activated T cells expressing a high-affinity IL-2 receptor (after previous exposure to IL-2) during proliferation are re-stimulated with antigen via the T cell receptor (TCR)/CD 3 complex, the expression of Fas ligand (FasL) and/or tumor necrosis factor (TNF) is induced, making the cells susceptible for Fas-mediated apoptosis. This process is IL-2-dependent and mediated via STATS. By the process of AICD in T lymphocytes tolerance can not only be established to self-antigens, but also to persistent antigens that are clearly not part of the host's makeup, such as tumor antigens.

For administration of fibroblasts, various protocols and procedures may be utilized. Guidance for administration of cell therapy in ALS may be derived from studies using various mesenchymal stem cell (MSC) approaches for this condition. For example, one of the first clinical interventions using mesenchymal stem cells in ALS was a report by Mazzini et al. [16], who treated ALS patients with bone marrow ex vivo expanded MSCs. Specifically, bone marrow collection was performed according to the standard procedure by aspiration from the posterior iliac crest. Ex vivo expansion of mesenchymal stem cells was induced according to Pittenger's protocol [17]. The cells were suspended in 2 ml of autologous cerebrospinal fluid and transplanted into the spinal cord by a micrometric pump injector. No patient manifested major adverse events such as respiratory failure or death. Minor adverse events were intercostal pain irradiation (4 patients), which was reversible after a mean period of three days after surgery, and leg sensory dysesthesia (5 patients), which was reversible after a mean period of six weeks after surgery. No modification of the spinal cord volume or other signs of abnormal cell proliferation were observed. The authors concluded by stating that it appears that the procedures of ex vivo expansion of autologous mesenchymal stem cells and of transplantation into the spinal cord of humans are safe and well tolerated by ALS patients. The same group reported a 3-year follow-up of the initial patients treated. Seven patients affected by definite ALS were enrolled in the study and two patients were treated for compassionate use. No patient manifested major adverse events such as respiratory failure or death. Minor adverse events were intercostal pain irradiation and leg sensory dysesthesia, both reversible after a mean period of 6 weeks. No modification of the spinal cord volume or other signs of abnormal cell proliferation were observed. A significant slowing down of the linear decline of the forced vital capacity was evident in four patients 36 months after MSC transplantation [18]. An additional two studies where performed by the same group on 10 and 19 patients. The longest observation of treated patients was performed at 9 years after treatment. No long-term adverse effects were detected and marginal therapeutic effects were seen [19, 20]. In another example, a study by an independent group evaluated the safety of two repeated intrathecal injections of autologous bone marrow (BM)-derived mesenchymal stromal cells (MSCs) in ALS patients. Eight patients with definite or probable ALS were enrolled. After a 3-month lead-in period, autologous MSCs were isolated two times from the BM at an interval of 26 days and were then expanded in vitro for 28 days and suspended in autologous cerebrospinal fluid. Of the 8 patients, 7 received 2 intrathecal injections of autologous MSCs (1×10(6) cells per kg) 26 days apart. Clinical or laboratory measurements were recorded to evaluate the safety 12 months after the first MSC injection. The ALS Functional Rating Scale-Revised (ALSFRS-R), the Appel ALS score, and forced vital capacity were used to evaluate the patients' disease status. One patient died before treatment and was withdrawn from the study. The death was not study related, and was attributable to natural progression of disease. With the exception of that patient, no serious adverse events were observed during the 12-month follow-up period. Most of the adverse events were self-limited or subsided after supportive treatment within 4 days. Decline in the ALSFRS-R score was not accelerated during the 6-month follow-up period. Two repeated intrathecal injections of autologous MSCs were safe and feasible throughout the duration of the 12-month follow-up period [21]. A subsequent study from Belarus utilized autologous mesenchymal stem cells injected intravenously (intact cells) or via lumbar puncture (cells committed to neuronal differentiation). Evaluation of the results of cell therapy after 12-month follow-up revealed slowing down of the disease progression, as assessed by ALSFRS-R score, was observed in 10 patients that were treated with cells. In comparison, in a control group that was matched for age and disease status, no slowing down of progression was observed. The Control group consisted of 15 patients. The study reported no adverse effects associated with administration of mesenchymal stem cells intravenously or intrathecally [22].

Other means of injection for administration are possible, such as intraventricular injection. A study by Baek et al. [23], assessed the ability to utilize intraventricular injections directly into the brain by using an Ommaya reservoir to administer cells. The Ommaya reservoir is a catheter system that is typically used for the delivery of drugs directly into the ventricles of the brain. It consists of a catheter in one lateral ventricle attached to a reservoir implanted under the scalp. It is typically used to treat brain tumors, leukemia/lymphoma or leptomeningeal disease, as well as for intracerebroventricular (ICV) injection of morphine [24]. Others have previously used the Ommaya reservoir to deliver cell therapy into the brain. To give an indication of the relative safety of this approach, in one study in glioma patients, autologous tumor infiltrating lymphocytes that were expanded ex vivo were administered in 6 patients by use of the Ommaya reservoir. One patient had complete response, 2 had partial responses, and 3 succumbed to disease. Most interestingly, no serious adverse effects were noted, despite the fact that activated lymphocytes were directly injected into the brain, an area typically classified as very sensitive in inflammation [25]. With the rational this, and other studies have successfully administered cells in the brain [26-28], and mesenchymal stem cells are generally considered anti-inflammatory, Baek et al. attempted to adopt this procedure for use in an ALS patient. Bone marrow mesenchymal stem cells were isolated from the bone marrow of a male patient with ALS who underwent insertion of an Ommaya reservoir. Expanded MSCs (hBM-MSCs: dose of 1×106 cells/kg) were suspended in autologous CSF and directly transplanted into the ALS patient's lateral ventricle via the Ommaya reservoir. Clinical, laboratory, and radiographic evaluation of the patient revealed no serious adverse effects related to the stem cell therapy. The authors concluded that intraventricular injection with an optimized number of cells is safe, and is a potential route for stem cell therapy in patients with ALS. Intraventricular injection via an Ommaya reservoir makes repetitive injection of stem cells easy and reliable even in far advanced ALS patients. Unfortunately, no discussion on impact on disease progression was given in the publication.

In another attempt to increase therapeutic efficacy of mesenchymal stem cells in ALS, researchers have explored in vitro means of augmenting neurotrophic factor production by manipulation of culture conditions, and these methods may be applied herein to fibroblasts of any kind. A series of studies from the Hadassah Medical Center in Jerusalem, Israel attempted to treat ALS by in vitro manipulated MSCs that are validated to produce higher amounts of neurotrophic factors. In the studies, all patients were followed up for 3 months before transplantation and 6 months after transplantation. In the phase ½ part of the trial, 6 patients with early-stage ALS were injected intramuscularly (IM) and 6 patients with more advanced disease were transplanted intrathecally (IT). In the second stage, a phase 2a dose-escalating study, 14 patients with early-stage ALS received a combined IM and IT transplantation of autologous MSC-NTF cells. It was reported that among the 12 patients in the phase ½ trial and the 14 patients in the phase 2a trial aged 20 and 75 years, the administration of mesenchymal stem cells was found to be safe and well tolerated over the study follow-up period. Most of the adverse effects were mild and transient, not including any treatment-related serious adverse event. The rate of progression of the forced vital capacity and of the ALSFRS-R score in the IT (or IT+IM)-treated patients was reduced (from −5.1% to −1.2%/month percentage predicted forced vital capacity, P<0.04 and from −1.2 to 0.6 ALSFRS-R points/month, P=0.052) during the 6 months following MSC-NTF cell transplantation vs. the pre-treatment period. Of these patients, 13 (87%) were defined as responders to either ALSFRS-R or forced vital capacity, having at least 25% improvement at 6 months after treatment in the slope of progression.

In some embodiments of the disclosure, well-known examples of approved drugs that augment endogenous neural stem cell activity include lithium [29, 30], valproic acid [31], and human chorionic gonadotropin [32] are utilized together with fibroblasts to inhibit and/or treat ALS. Interestingly, the stem cell modifier combination of lithium and valproic acid was already assessed on its own in a small trial which suggested some possible efficacy. The study recruited 18 patients that were treated with the combination and compared them to 31 controls that were carefully paired by age, gender, evolution rate and time of the disease, who never received treatment with lithium and/or valproate. Assessment of disease by ALSFRS-R was performed before treatment (baseline), 1 month after treatment, and every 4 months until the outcome (death or an adverse event). The investigators reported that lithium and valproate co-treatment significantly increased survival, and this treatment also exerted neuroprotection in the patients because all biochemical markers reached normal levels in the ALS patients that were treated. The biochemical markers were Cu/Zn superoxide dismutase and glutathione peroxidase activity, and reduced glutathione [33].

In one embodiment of the disclosure, patients suffering from ALS or at risk for ALS are administered with 0.3×106 IU of IL-2 (such as aldesleukin) daily after administration of 10,000-4,000,000 million fibroblasts per kilogram of body weight. Concentrations for clinical uses of IL-2 (including aldesleukin) could be used from the literature as described for other indications including heart failure [15], Wiskott-Aldrich syndrome [34], Graft Versus Host Disease [35, 36], lupus [37], type 1 diabetes [38-40] and are incorporated by reference. In some embodiments of the disclosure, administration of low doses of IL-2, such as in the form of aldesleukin, every day at concentrations of 0.3×106 to 3.0×106 IU IL-2 per square meter of body surface area for 8 weeks, or in other embodiments repetitive 5-day courses of 1.0×106 to 3.0×106 IU IL-2. Various types of IL-2 may be utilized. Examples of IL-2 variants, recombinant IL-2, methods of IL-2 production, methods of IL-2 purification, methods of formulation, and the like are well known in the art and can be found, for example, at least in U.S. Pat. Nos. 4,530,787, 4,569,790, 4,572,798, 4,604,377, 4,748,234, 4,853,332, 4,959,314, 5,464,939, 5,229,109, 7,514,073, and 7,569,215, each of which is herein incorporated by reference in their entirety for all purposes. In some embodiments, low dose interleukin-2 is provided together with one or more activators of coinhibitory molecules, otherwise known as checkpoints. Such coinhibitory molecules include CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, A2aR, and combinations thereof. In some embodiments of the disclosure, mesenchymal stem cells are co-administered. Protocols for use of MSC have been previously published and incorporated by reference [41, 42]. For example, mesenchymal stem cells of adipose [43-46], bone marrow [47-66], placental [67], amniotic membrane [68, 69], umbilical cord [70-76], menstrual blood [77], and lung [78, 79], origin, as well as conditioned media [80-87]. Additionally, the generation of Treg by mesenchymal stem cells is also described in the art, for which we are providing the following references to assist in the practice of the invention [88-116].

In certain embodiments, patients with ALS are administered human IL-2 muteins that preferentially stimulate T regulatory (Treg) cells. As used herein “preferentially stimulates T regulatory cells” means the mutein promotes the proliferation, survival, activation and/or function of CD3+FoxP3+ T cells over CD3+FoxP3− T cells. Methods of measuring the ability to preferentially stimulate Tregs can be measured by flow cytometry of peripheral blood leukocytes, in which there is an observed increase in the percentage of FOXP3+CD4+ T cells among total CD4+ T cells, an increase in percentage of FOXP3+CD8+ T cells among total CD8+ T cells, an increase in percentage of FOXP3+ T cells relative to NK cells, and/or a greater increase in the expression level of CD25 on the surface of FOXP3+ T cells relative to the increase of CD25 expression on other T cells. Preferential growth of Treg cells can also be detected as increased representation of demethylated FOXP3 promoter DNA (i.e. the Treg-specific demethylated region, or TSDR) relative to demethylated CD3 genes in DNA extracted from whole blood, as detected by sequencing of polymerase chain reaction (PCR) products from bisulfite-treated genomic DNA. IL-2 muteins that preferentially stimulate Treg cells increase the ratio of CD3+FoxP3+ T cells over CD3+FoxP3− T cells in a subject or a peripheral blood sample at least 30%, at least 40%, at least 50%, at least 60%, at least 70%, at least 80%, at least 90%, at least 100%, at least 150%, at least 200%, at least 300%, at least 400%, at least 500%, at least 600%, at least 700%, at least 800%, at least 900%, or at least 1000%.

In some embodiments of the disclosure, patients suffering from ALS are administered with agents which possess properties capable of enhancing T regulatory cells through stimulation of mesenchymal stem cells administered in an allogeneic host. Proteins, such as antibodies, fusion proteins, and soluble ligands, any of which may either be identical to a wild-type protein or contain a mutation (i.e., a deletion, addition, or substitution of one or more amino acid residues), and the nucleic acid molecules that encode them (or that are “antisense” to them; e.g., an oligonucleotide that is antisense to the nucleic acids that encode a target polypeptide, or a component (e.g., a subunit) of their receptors), are all “agents.” The agents of the invention can either be administered systemically, locally, or by way of cell-based therapies (i.e., an agent of the invention can be administered to a patient by administering a cell that expresses that agent to the patient). A tolerance restoring agent can be alpha1-antitrypsin (AAT; sometimes abbreviated A1AT), which is also referred to as alpha1-proteinase inhibitor. AAT is a major serum serine-protease inhibitor that inhibits the enzymatic activity of numerous serine proteases including neutrophil elastase, cathepsin G, proteinase 3, thrombin, trypsin and chymotrypsin. For example, one can administer an AAT polypeptide (e.g., a purified or recombinant AAT, such as human AAT) or a homolog, biologically active fragment, or other active mutant thereof. alpha1 proteinase inhibitors are commercially available for the treatment of AAT deficiencies, and include ARALAST™, PROLASTIN™ and ZEMAIRA™. The AAT polypeptide or the biologically active fragment or mutant thereof can be of human origin and can be purified from human tissue or plasma. Alternatively, it can be recombinantly produced. For ease of reading, we do not repeat the phrase “or a biologically active fragment or mutant thereof” after each reference to AAT. It is to be understood that, whenever a full-length, naturally occurring AAT can be used, a biologically active fragment or other biologically active mutant thereof (e.g., a mutant in which one or more amino acid residues have be substituted) can also be used. Similarly, we do not repeat on each occasion that a naturally occurring polypeptide (e.g., AAT) can be purified from a natural source or recombinantly produced. It is to be understood that both forms may be useful. Similarly, we do not repeatedly specify that the polypeptide can be of human or non-human origin. While there may be advantages to administering a human protein, the invention is not so limited.

The methods of the present disclosure (e.g., multiple-variable dose IL-2 alone or in combination with one or more other anti-immune disorder therapies) can be administered to a desired subject or once a subject is indicated as being a likely responder to such therapy. In another embodiment, the therapeutic methods of the present invention can be avoided if a subject is indicated as not being a likely responder to the therapy and an alternative treatment regimen, such as targeted and/or untargeted anti-immune therapies, can be administered.

In one embodiment, a multiple-variable IL-2 dose method of treating a subject suffering from ALS a therapy comprising a) administering to the subject an induction regimen comprising continuously administering to the subject interleukin-2 (IL-2) at a dose that increases the subject's plasma IL-2 level and increases the subject's ratio of immune suppressive T cells to conventional T lymphocytes (Tcons) and b) subsequently administering to the subject at least one maintenance regimen comprising continuously administering to the subject an IL-2 maintenance dose that is higher than the induction regimen dose and that i) further increases the subject's plasma IL-2 level and ii) further increases the ratio of immune suppressive T cells to Tcons, thereby treating the subject, is provided. In one embodiment, the level of plasma IL-2 resulting from the induction regimen is depleted below that of the prior peak plasma IL-2 level before the induction regimen. The IL-2 maintenance regimen can, in certain embodiments, increase the subject's plasma IL-2 level beyond the peak plasma IL-2 level induced by the induction regimen. The term “multiple-variable IL-2 dose method” refers to a therapeutic intervention comprising more than one IL-2 administration, wherein the more than one IL-2 administration uses more than one IL-2 dose. Such a method is contrasted from a “fixed” dosed method wherein a fixed amount of IL-2 is administered in a scheduled manner, such as daily. The term “induction regimen” refers to the continuous administration of IL-2 at a dose that increases the subject's plasma IL-2 level and increases the subject's immune suppressive T cells:Tcons ratio. In some embodiments, the regimen occurs until a peak level of plasma IL-2 is achieved. The subject's plasma IL-2 level and/or immune suppressive T cell:Tcons ratio can be increased by at least 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200% or more relative to the baseline ratio prior to initiation of therapy.

Low-dose IL-2 may be utilized, and the term “low-dose IL-2” refers to the dosage range wherein immune suppressive T cells are preferentially enhanced relative to Tcons. In one embodiment, low-dose IL-2 refers to IL-2 doses that are less than or equal to 50% of the “high-dose IL-2” doses (e.g., 18 million IU per m2 per day to 20 million IU per m2 per day, or more) used for anti-cancer immunotherapy. The upper limit of “low-dose IL-2” can further be limited by treatment adverse events, such as fever, chills, asthenia, and fatigue. IL-2 is generally dosed according to an amount measured in international units (IU) administered in comparison to body surface area (BSA) per given time unit. BSA can be calculated by direct measurement or by any number of well-known methods (e.g., the Dubois & Dubois formula), such as those described in the Examples. Generally, IL-2 is administered according in terms of IU per m2 of BSA per day. Exemplary low-dose IL-2 doses according to the methods of the present invention include, in terms of 106 IU/m2/day, any one of 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, and 3.0×106 IU/m2/day, including any values in between and/or ranges in between. For example, an induction regimen dose can range between 0.3×106 IU/m2/day and 3.0×106 IU/m2/day with any value or range in between.

Continuous administration may be utilized, and the term “continuous administration” refers to administration of IL-2 at regular intervals without any intermittent breaks in between. Thus, no interruptions in IL-2 occur. For example, the induction dose can be administered every day (e.g., once or more per day) during at least 1-14 consecutive days or any range in between (e.g., at least 4-7 consecutive days). As described herein, longer acting IL-2 agents and/or IL-2 agents administered by routes other than subcutaneous administration are contemplated. Intermittent intravenous administration of IL-2 described in the art results in short IL-2 half lives incompatible with increasing plasma IL-2 levels and increasing the immune suppressive T cells:Tcons ratio according to the present invention. However, once-daily subcutaneous IL-2 dosing, continuous IV infusion, long-acting subcutaneous IL-2 formulations, and the like are contemplated for achieving a persistent steady state IL-2 level.

As described above, IL-2 can be administered in a pharmaceutically acceptable formulation and by any suitable administration route, such as by subcutaneous, intravenous, intraperitoneal, oral, nasal, transdermal, or intramuscular administration. In one embodiment, the present invention provides pharmaceutically acceptable compositions which compose IL-2 at a therapeutically-effective amount, formulated together with one or more pharmaceutically acceptable carriers (additives) and/or diluents. The pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

In some embodiments of the disclosure, a monoclonal antibody (mAb) against the CD3 molecule is utilized, such as for immune modulation of the ALS patient together with IL-2 or without IL-2 and/or with fibroblasts. This approach has previously been used to induced tolerance to autoimmunity in murine models of type 1 diabetes mellitus. Treatment with anti-CD3 mAb reversed diabetes in the NOD mouse and prevented recurrent immune responses toward transplanted syngeneic islets. This was achieved without the need for continuous immune suppression and persisted at a time when T cell numbers were not depleted and were quantitatively normal. Another approach is to induce specific immunological unresponsiveness by administering self-antigens.

An example of how different CD3 targeting antibodies can elicit different effects is seen in another study, which Davis et al. examined the IgM monoclonal antibody called 38.1, which was distinct from other anti-CD3 mAb, in that it was rapidly modulated from the cell surface in the absence of a secondary antibody. Although 38.1 induced an immediate increase in intracellular free calcium [Ca2+]i by highly purified T cells, it did not induce entry of the cells into the cell cycle in the absence of accessory cells (AC) or a protein kinase C-activating phorbol ester. Treated T cells were markedly inhibited in their capacity to respond to the T cell stimulating mitogen phytohemagluttanin. Inhibition of responsiveness could be overcome by culturing the cells with supplemental antigen presenting cells or the cytokine IL-2. These studies demonstrate that a state of T cell nonresponsiveness can be induced by modulating CD3 with an anti-CD3 mAb in the absence of co-stimulatory signals. A brief increase in [Ca2+]i resulting from mobilization of internal calcium stores appears to be sufficient to induce this state of T cell nonresponsiveness [117].

In some situations, anti-CD3 antibodies have been shown to program T cells towards antigen-specific tolerance. This is illustrated in one example in the work of Anasetti et al. who exposed PBMC to alloantigen for 3-8 d in the presence of anti-CD3 antibodies. They showed no response after restimulation with cells from the original donor but the PBMC remained capable of responding to third-party donors. Antigen-specific nonresponsiveness was induced by both nonmitogenic and mitogenic anti-CD3 antibodies but not by antibodies against CD2, CD4, CD5, CD8, CD18, or CD28. This suggested the unique ability of this protein to modulate programs in the T cells that are antigen specific. Nonresponsiveness induced by anti-CD3 antibody in mixed leukocyte culture was sustained for at least 34 d from initiation of the culture and 26 d after removal of the antibody. Anti-CD3 antibody also induced antigen-specific nonresponsiveness in cytotoxic T cell generation assays. Anti-CD3 antibody did not induce nonresponsiveness in previously primed cells [118].

The use of anti-CD3 antibodies for the practice of the embodiments of the disclosure encompasses that the antibodies not only do not result in activation of T cell proliferation and inflammatory cytokine secretion, but also that the T cells actually inhibit inflammation and promote regeneration.

In one embodiment of the disclosure, anti-CD3 antibody is given 14 days before administration of mesenchymal stem cells In one specific embodiment, said 14-day course of the anti-CD3 monoclonal antibody utilizes the antibody hOKT3γ1 (Ala-Ala) administered intravenously (1.42 μg per kilogram of body weight on day 1; 5.67 μg per kilogram on day 2; 11.3 μg per kilogram on day 3; 22.6 μg per kilogram on day 4; and 45.4 μg per kilogram on days 5 through 14); these doses were based on those previously used for treatment of transplant rejection [119] which is incorporated by reference. Other types of anti-CD3 molecules and dosing regimens may be used in the context of ALS therapeutics, said doses may be chosen from examples of utility of anti-CD3 from the literature, as described in the following papers and incorporated by reference: prevention of kidney [120-128], liver [129-131], pancreas [132-134], lung [135], and heart [136-140] transplant rejection; prevention of graft versus host disease [141], multiple sclerosis [142], type 1 diabetes [143],

The use of monoclonal antibodies for the practice of the invention must be tempered by the caution that in some cases cytokine storm may be initiated by antibody administration [144, 145]. In some cases this is concentration dependent [146]. Treatment for this can be accomplished by steroid administration or anti-IL6 antibody [147-151].

In some embodiments of the disclosure, administration of PGE1 and/or various natural anti-inflammatory compounds are provided to decrease TNF-alpha production as a result of anti-CD3 administration, such as described in this paper and incorporated by reference [152]. In further embodiments of the invention, administration of anti-CD3 may be performed together with endothelial protectants and/or anti-coagulants in order to reduce clotting associated with CD3 modulating agents [153]. In some embodiments anti-CD3 antibodies may be used in combination with tolerogenic cytokines such as interleukin-10 in order to enhance number of angiogenesis supporting T cells. The safety of anti-CD3 and IL-10 administration has previously been demonstrated in a clinical trial [154].

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate butler solutions; and (21) other non-toxic compatible substances employed in pharmaceutical formulations. Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

In one embodiment, the Treg cell surface protein is selected from the group consisting of CD25, GITR, TIGIT, CTLA-4, neuropilin, OX40, LAG3, and combinations thereof, said Tregs are isolated possessing said surfaces proteins from a tissue source, and optionally expanded ex vivo prior to administration to a patient suffering from ALS.

In one embodiment of the disclosure, utilization of extracorporeal manipulations is used to generate an environment suitable of T regulatory survival after administration from exogenous sources, or to enhance survival of endogenous T regulatory cells. The extracorporeal removal of various physiological or pathological agents has been part of medical practice since the development of renal dialysis in the late 1940s by William Kolff [155]. Advanced means of extracorporeal removal of various substances has been demonstrated in the case of immune complex removal [156-159], antibodies [160-165], viruses [166-168], soluble receptors [169], and even cells [170, 171]. These methodologies may be used to optimize efficacy of the current invention to remove T regulatory cell inhibitory compounds such as TNF-alpha, Interferon gamma, or interleukin-33 intra alia.

In some embodiments stimulators of HGF are add to enhance proliferation of T regulatory cells [172-175].

In one embodiment, the disclosure teaches the use of activation of fibroblasts prior to therapeutic use, and/or administration of agents which act as “regenerative adjuvants” for said fibroblasts. The cells in the formulation display typical fibroblast morphologies when growing in cultured monolayers. Specifically, cells may display an elongated, fusiform or spindle appearance with slender extensions, or cells may appear as larger, flattened stellate cells which may have cytoplasmic leading edges. A mixture of these morphologies may also be observed. The cells express proteins characteristic of normal fibroblasts including the fibroblast-specific marker, CD90 (Thy-1), a 35 kDa cell-surface glycoprotein, and the extracellular matrix protein, collagen. The fibroblast dosage formulation is an autologous cell therapy product composed of a suspension of autologous fibroblasts, grown from a biopsy of each individual's own skin using standard tissue culture procedures. In one embodiment the fibroblasts of the invention can also be used to create other cell types for tissue repair or regeneration.

The fibroblasts utilized in the disclosure are generated, in one embodiment, by outgrowth from a biopsy of the recipient's own skin (in the case of autologous preparations), or skin of healthy donors (for allogeneic preparations). In some embodiments fibroblasts are used from young donors. In another embodiment fibroblasts are transfected with genes to allow for enhanced growth and overcoming of the Hayflick limit. Subsequent to derivation of cells expansion in culture using standard cell culture techniques. Skin tissue (dermis and epidermis layers) may be biopsied from a subject's post-auricular area. In one embodiment, the starting material is composed of three 3-mm punch skin biopsies collected using standard aseptic practices. The biopsies are collected by the treating physician, placed into a vial containing sterile phosphate buffered saline (PBS). The biopsies are shipped in a 2-8° C. refrigerated shipper back to the manufacturing facility. In one embodiment, after arrival at the manufacturing facility, the biopsy is inspected and, upon acceptance, transferred directly to the manufacturing area. Upon initiation of the process, the biopsy tissue is then washed prior to enzymatic digestion. After washing, a Liberase Digestive Enzyme Solution is added without mincing, and the biopsy tissue is incubated at 37.0.+−0.2° C. for one hour. Time of biopsy tissue digestion is a critical process parameter that can affect the viability and growth rate of cells in culture. Liberase is a collagenase/neutral protease enzyme cocktail obtained formulated from Lonza Walkersville, Inc. (Walkersville, Md.) and unformulated from Roche Diagnostics Corp. (Indianapolis, Ind.). Alternatively, other commercially available collagenases may be used, such as Serva Collagenase NB6 (Helidelburg, Germany). After digestion, Initiation Growth Media (IMDM, GA, 10% Fetal Bovine Serum (FBS)) is added to neutralize the enzyme, cells are pelleted by centrifugation and resuspended in 5.0 mL Initiation Growth Media. Alternatively, centrifugation is not performed, with full inactivation of the enzyme occurring by the addition of Initiation Growth Media only. Initiation Growth Media is added prior to seeding of the cell suspension into a T-175 cell culture flask for initiation of cell growth and expansion. A T-75, T-150, T-185 or T-225 flask can be used in place of the T-75 flask. Cells are incubated at 37.+−0.2.0° C. with 5.0.+−0.1.0% CO2 and fed with fresh Complete Growth Media every three to five days. All feeds in the process are performed by removing half of the Complete Growth Media and replacing the same volume with fresh media. Alternatively, full feeds can be performed. Cells should not remain in the T-175 flask greater than 30 days prior to passaging. Confluence is monitored throughout the process to ensure adequate seeding densities during culture splitting. When cell confluence is greater than or equal to 40% in the T-175 flask, they are passaged by removing the spent media, washing the cells, and treating with Trypsin-EDTA to release adherent cells in the flask into the solution. Cells are then trypsinized and seeded into a T-500 flask for continued cell expansion. Alternately, one or two T-300 flasks, One Layer Cell Stack (1 CS), One Layer Cell Factory (1 CF) or a Two Layer Cell Stack (2 CS) can be used in place of the T-500 Flask. Morphology is evaluated at each passage and prior to harvest to monitor the culture purity throughout the culture purity throughout the process. Morphology is evaluated by comparing the observed sample with visual standards for morphology examination of cell cultures. The cells display typical fibroblast morphologies when growing in cultured monolayers. Cells may display either an elongated, fusiform or spindle appearance with slender extensions, or appear as larger, flattened stellate cells which may have cytoplasmic leading edges. A mixture of these morphologies may also be observed. Fibroblasts in less confluent areas can be similarly shaped, but randomly oriented. The presence of keratinocytes in cell cultures is also evaluated. Keratinocytes appear round and irregularly shaped and, at higher confluence, they appear organized in a cobblestone formation. At lower confluence, keratinocytes are observable in small colonies. Cells are incubated at 37.+−0.2.0° C. with 5.0.+−0.1.0% CO2 and passaged every three to five days in the T-500 flask and every five to seven days in the ten layer cell stack (10CS). Cells should not remain in the T-500 flask for more than 10 days prior to passaging. Quality Control (QC) release testing for safety of the Bulk Drug Substance includes sterility and endotoxin testing. When cell confluence in the T-500 flask is .gtoreq.95%, cells are passaged to a 10 CS culture vessel. Alternately, two Five Layer Cell Stacks (5 CS) or a 10 Layer Cell Factory (10 CF) can be used in place of the 10 CS. 10CS. Passage to the 10 CS is performed by removing the spent media, washing the cells, and treating with Trypsin-EDTA to release adherent cells in the flask into the solution. Cells are then transferred to the 10 CS. Additional Complete Growth Media is added to neutralize the trypsin and the cells from the T-500 flask are pipetted into a 2 L bottle containing fresh Complete Growth Media. The contents of the 2 L bottle are transferred into the 10 CS and seeded across all layers. Cells are then incubated at 37.+−0.2.0° C. with 5.0.+−0.1.0% CO2 and fed with fresh Complete Growth Media every five to seven days. Cells should not remain in the 10CS for more than 20 days prior to passaging. In one embodiment, the passaged dermal fibroblasts are rendered substantially free of immunogenic proteins present in the culture medium by incubating the expanded fibroblasts for a period of time in protein free medium, Primary Harvest When cell confluence in the 10 CS is 95% or more, cells are harvested. Harvesting is performed by removing the spent media, washing the cells, treating with Trypsin-EDTA to release adherent cells into the solution, and adding additional Complete Growth Media to neutralize the trypsin. Cells are collected by centrifugation, resuspended, and in-process QC testing performed to determine total viable cell count and cell viability.

The therapy provided herein may comprise administration of a therapeutic agents (e.g., fibroblasts, exosomes from fibroblasts, etc.) alone or in combination. Therapies may be administered in any suitable manner known in the art. For example, a first and second treatment may be administered sequentially (at different times) or concurrently (at the same time). In some embodiments, the first and second treatments are administered in a separate composition. In some embodiments, the first and second treatments are in the same composition. Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.] The therapeutic agents (e.g., fibroblasts) of the disclosure may be administered by the same route of administration or by different routes of administration. In some embodiments, the therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some embodiments, between about 105 and about 1013 cells per 100 kg are administered to a human per infusion. In some embodiments, between about 1.5×106 and about 1.5×1012 cells are infused per 100 kg. In some embodiments, between about 1×109 and about 5×1011 cells are infused per 100 kg. In some embodiments, between about 4×109 and about 2×1011 cells are infused per 100 kg. In some embodiments, between about 5×108 cells and about 1×1012 cells are infused per 100 kg. In some embodiments, a single administration of cells is provided. In some embodiments, multiple administrations are provided. In some embodiments, multiple administrations are provided over the course of 3-7 consecutive days. In some embodiments, 3-7 administrations are provided over the course of 3-7 consecutive days. In some embodiments, 5 administrations are provided over the course of 5 consecutive days. In some embodiments, a single administration of between about 105 and about 1013 cells per 100 kg is provided. In some embodiments, a single administration of between about 1.5×108 and about 1.5×1012 cells per 100 kg is provided. In some embodiments, a single administration of between about 1×109 and about 5×1011 cells per 100 kg is provided. In some embodiments, a single administration of about 5×1010 cells per 100 kg is provided. In some embodiments, a single administration of 1×1010 cells per 100 kg is provided. In some embodiments, multiple administrations of between about 105 and about 1013 cells per 100 kg are provided. In some embodiments, multiple administrations of between about 1.5×108 and about 1.5×1012 cells per 100 kg are provided. In some embodiments, multiple administrations of between about 1×109 and about 5×1011 cells per 100 kg are provided over the course of 3-7 consecutive days. In some embodiments, multiple administrations of about 4×109 cells per 100 kg are provided over the course of 3-7 consecutive days. In some embodiments, multiple administrations of about 2×1011 cells per 100 kg are provided over the course of 3-7 consecutive days. In some embodiments, 5 administrations of about 3.5×109 cells are provided over the course of 5 consecutive days. In some embodiments, 5 administrations of about 4×109 cells are provided over the course of 5 consecutive days. In some embodiments, 5 administrations of about 1.3×1011 cells are provided over the course of 5 consecutive days. In some embodiments, 5 administrations of about 2×1011 cells are provided over the course of 5 consecutive days.

In one embodiment, fibroblasts are cultured using means known in the art for preserving viability and proliferative ability of fibroblasts. The invention may be applied both for individualized autologous exosome preparations and for exosome preparations obtained from established cell lines, for experimental or biological use. In one embodiment, this invention is more specifically based on the use of chromatography separation methods for preparing membrane vesicles, particularly to separate the membrane vesicles from potential biological contaminants, wherein said microvesicles are exosomes, and cells utilized for generating said exosomes are fibroblast cells.

Indeed, the applicant has now demonstrated that membrane vesicles, particularly exosomes, could be purified, and possess ability to inhibit pain. In one embodiment, a strong or weak, preferably strong, anion exchange may be performed. In addition, in a specific embodiment, the chromatography is performed under pressure. Thus, more specifically, it may consist of high performance liquid chromatography (HPLC). Different types of supports may be used to perform the anion exchange chromatography. More preferably, these may include cellulose, poly(styrene-divinylbenzene), agarose, dextran, acrylamide, silica, ethylene glycol-methacrylate co-polymer, or mixtures thereof, e.g., agarose-dextran mixtures. To illustrate this, it is possible to mention the different chromatography equipment composed of supports as mentioned above, particularly the following gels: POROS®. SEPHAROSE®, SEPHADEX®, TRISACRYL®, TSK-GEL SW OR PW®, SUPERDEX® and SEPHACRYL®, for example, which are suitable for the application of this invention. Therefore, in a specific embodiment, this disclosure relates to a method of preparing membrane vesicles, particularly exosomes, from a biological sample such as a tissue culture containing fibroblasts, comprising at least one step during which the biological sample is treated by anion exchange chromatography on a support selected from cellulose, poly(styrene-divinylbenzene), silica, acrylamide, agarose, dextran, ethylene glycol-methacrylate co-polymer, alone or in mixtures, optionally functionalized.

In addition, to improve the chromatographic resolution, within the scope of the invention, it is preferable to use supports in bead form. Ideally, these beads have a homogeneous and calibrated diameter, with a sufficiently high porosity to enable the penetration of the objects under chromatography (i.e. the exosomes). In this way, given the diameter of exosomes (generally between 50 and 100 nm), to apply the invention, it is preferable to use high porosity gels, particularly between 10 nm and 5.mu·m, more preferably between approximately 20 nm and approximately 2.mu·m, even more preferably between about 100 nm and about 1.mu·m. For the anion exchange chromatography, the support used must be functionalised using a group capable of interacting with an anionic molecule. Generally, this group is composed of an amine which may be ternary or quaternary, which defines a weak or strong anion exchanger, respectively. Within the scope of this invention, it is particularly advantageous to use a strong anion exchanger. In this way, according to the invention, a chromatography support as described above, functionalised with quaternary amines, is used. Therefore, according to a more specific embodiment of the invention, the anion exchange chromatography is performed on a support functionalised with a quaternary amine. Even more preferably, this support should be selected from poly(styrene-divinylbenzene), acrylamide, agarose, dextran and silica, alone or in mixtures, and functionalised with a quaternary amine. Examples of supports functionalised with a quaternary amine include the gels SOURCEQ. MONO Q, Q SEPHAROSE®, POROS® HQ and POROS® QE, FRACTOGEL® TMAE type gels and TOYOPEARL SUPER® Q gels.

One support to perform the anion exchange chromatography comprises poly(styrene-divinylbenzene). An example of this type of gel which may be used within the scope of this invention is SOURCE Q gel, particularly SOURCE 15 Q (Pharmacia). This support offers the advantage of very large internal pores, thus offering low resistance to the circulation of liquid through the gel, while enabling rapid diffusion of the exosomes to the functional groups, which are particularly important parameters for exosomes given their size. The biological compounds retained on the column may be eluted in different ways, particularly using the passage of a saline solution gradient of increasing concentration, e.g. from 0 to 2 M. A sodium chloride solution may particularly be used, in concentrations varying from 0 to 2 M, for example. The different fractions purified in this way are detected by measuring their optical density (OD) at the column outlet using a continuous spectro-photometric reading. As an indication, under the conditions used in the examples, the fractions comprising the membrane vesicles were eluted at an ionic strength comprised between approximately 350 and 700 mM, depending on the type of vesicles.

Different types of columns may be used to perform this chromatographic step, according to requirements and the volumes to be treated. For example, depending on the preparations, it is possible to use a column from approximately 100.mu·l up to 10 ml or greater. In this way, the supports available have a capacity which may reach 25 mg of proteins/ml, for example. For this reason, a 100.mu·l column has a capacity of approximately 2.5 mg of proteins which, given the samples in question, allows the treatment of culture supernatants of approximately 2.1 (which, after concentration by a factor of 10 to 20, for example, represent volumes of 100 to 200 ml per preparation). It is understood that higher volumes may also be treated, by increasing the volume of the column, for example. In addition, to perform this invention, it is also possible to combine the anion exchange chromatography step with a gel permeation chromatography step. In this way, according to a specific embodiment of the invention, a gel permeation chromatography step is added to the anion exchange step, either before or after the anion exchange chromatography step. Preferably, in this embodiment, the permeation chromatography step takes place after the anion exchange step. In addition, in a specific variant, the anion exchange chromatography step is replaced by the gel permeation chromatography step. The present application demonstrates that membrane vesicles may also be purified using gel permeation liquid chromatography, particularly when this step is combined with an anion exchange chromatography or other treatment steps of the biological sample, as described in detail below.

To perform the gel permeation chromatography step, a support selected from silica, acrylamide, agarose, dextran, ethylene glycol-methacrylate co-polymer or mixtures thereof, e.g., agarose-dextran mixtures, are preferably used. As an illustration, for gel permeation chromatography, a support such as SUPERDEX® 200HR (Pharmacia), TSK G6000 (TosoHaas) or SEPHACRYL® S (Pharmacia) is preferably used. The process according to the invention may be applied to different biological samples. In particular, these may consist of a biological fluid from a subject (bone marrow, peripheral blood, etc.), a culture supernatant, a cell lysate, a pre-purified solution or any other composition comprising membrane vesicles.

In this respect, in a specific embodiment of the invention, the biological sample is a culture supernatant of membrane vesicle-producing fibroblast cells.

In addition, according to a preferred embodiment of the invention, the biological sample is treated, prior to the chromatography step, to be enriched with membrane vesicles (enrichment stage). In this way, in a specific embodiment, this invention relates to a method of preparing membrane vesicles from a biological sample, characterised in that it comprises at least: b) an enrichment step, to prepare a sample enriched with membrane vesicles, and c) a step during which the sample is treated by anion exchange chromatography and/or gel permeation chromatography.

In one embodiment, the biological sample is a culture supernatant treated so as to be enriched with membrane vesicles. In particular, the biological sample may be composed of a pre-purified solution obtained from a culture supernatant of a population of membrane vesicle-producing cells or from a biological fluid, by treatments such as centrifugation, clarification, ultrafiltration, nanofiltration and/or affinity chromatography, particularly with clarification and/or ultrafiltration and/or affinity chromatography. Therefore, a preferred method of preparing membrane vesicles according to this invention more particularly comprises the following steps: a) culturing a population of membrane vesicle (e.g. exosome) producing cells under conditions enabling the release of vesicles, b) a step of enrichment of the sample in membrane vesicles, and c) an anion exchange chromatography and/or gel permeation chromatography treatment of the sample.

As indicated above, the sample (e.g. supernatant) enrichment step may comprise one or more centrifugation, clarification, ultrafiltration, nanofiltration and/or affinity chromatography steps on the supernatant. In a first specific embodiment, the enrichment step comprises (i) the elimination of cells and/or cell debris (clarification), possibly followed by (ii) a concentration and/or affinity chromatography step. In another specific embodiment, the enrichment step comprises an affinity chromatography step, optionally preceded by a step of elimination of cells and/or cell debris (clarification). A preferred enrichment step according to this invention comprises (i) the elimination of cells and/or cell debris (clarification), (ii) a concentration and (iii) an affinity chromatography. The cells and/or cell debris may be eliminated by centrifugation of the sample, for example, at a low speed, preferably below 1000 g, between 100 and 700 g, for example. Preferred centrifugation conditions during this step are approximately 300 g or 600 g for a period between 1 and 15 minutes, for example.

The cells and/or cell debris may also be eliminated by filtration of the sample, possibly combined with the centrifugation described above. The filtration may particularly be performed with successive filtrations using filters with a decreasing porosity. For this purpose, filters with a porosity above 0.2 mm, e.g. between 0.2 and 10 mm, are preferentially used. It is particularly possible to use a succession of filters with a porosity of 10 mm, 1 mm, 0.5 mm followed by 0.22 mm.

A concentration step may also be performed, in order to reduce the volumes of sample to be treated during the chromatography stages. In this way, the concentration may be obtained by centrifugation of the sample at high speeds, e.g. between 10,000 and 100,000 g, to cause the sedimentation of the membrane vesicles. This may consist of a series of differential centrifugations, with the last centrifugation performed at approximately 70,000 g. The membrane vesicles in the pellet obtained may be taken up with a smaller volume and in a suitable buffer for the subsequent steps of the process. The concentration step may also be performed by ultrafiltration. In fact, this ultrafiltration allows both to concentrate the supernatant and perform an initial purification of the vesicles. According to a preferred embodiment, the biological sample (e.g., the supernatant) is subjected to an ultrafiltration, preferably a tangential ultrafiltration. Tangential ultrafiltration consists of concentrating and fractionating a solution between two compartments (filtrate and retentate), separated by membranes of determined cut-off thresholds. The separation is carried out by applying a flow in the retentate compartment and a transmembrane pressure between this compartment and the filtrate compartment. Different systems may be used to perform the ultrafiltration, such as spiral membranes (Millipore, Amicon), flat membranes or hollow fibres (Amicon, Millipore, Sartorius, Pall, GF, Sepracor). Within the scope of the invention, the use of membranes with a cut-off threshold below 1000 kDa, preferably between 300 kDa and 1000 kDa, or even more preferably between 300 kDa and 500 kDa, is advantageous.

The affinity chromatography step can be performed in various ways, using different chromatographic support and material. It is advantageously a non-specific affinity chromatography, aimed at retaining (i.e., binding) certain contaminants present within the solution, without retaining the objects of interest (i.e., the exosomes). It is therefore a negative selection. Preferably, an affinity chromatography on a dye is used, allowing the elimination (i.e., the retention) of contaminants such as proteins and enzymes, for instance albumin, kinases, deshydrogenases, clotting factors, interferons, lipoproteins, or also co-factors, etc. More preferably, the support used for this chromatography step is a support as used for the ion exchange chromatography, functionalised with a dye. As specific example, the dye may be selected from Blue SEPHAROSE® (Pharmacia), YELLOW 86, GREEN 5 and BROWN 10 (Sigma). The support is more preferably agarose. It should be understood that any other support and/or dye or reactive group allowing the retention (binding) of contaminants from the treated biological sample can be used in the instant disclosure.

In one embodiment a membrane vesicle preparation process within the scope of this disclosure comprises the following steps: a) the culture of a population of membrane vesicle (e.g. exosome) producing cells under conditions enabling the release of vesicles, b) the treatment of the culture supernatant with at least one ultrafiltration or affinity chromatography step, to produce a biological sample enriched with membrane vesicles (e.g. with exosomes), and c) an anion exchange chromatography and/or gel permeation chromatography treatment of the biological sample. In a preferred embodiment, step b) above comprises a filtration of the culture supernatant, followed by an ultrafiltration, preferably tangential. In another preferred embodiment, step b) above comprises a clarification of the culture supernatant, followed by an affinity chromatography on dye, preferably on Blue SEPHAROSE®.

In addition, after step c), the material harvested may, if applicable, be subjected to one or more additional treatment and/or filtration stages d), particularly for sterilisation purposes. For this filtration treatment stage, filters with a diameter less than or equal to 0.3.mu·m are preferentially used, or even more preferentially, less than or equal to 0.25.mu·m. Such filters have a diameter of 0.22.mu·m, for example. After step d), the material obtained is, for example, distributed into suitable devices such as bottles, tubes, bags, syringes, etc., in a suitable storage medium. The purified vesicles obtained in this way may be stored cold, frozen or used extemporaneously. Therefore, a specific preparation process within the scope of the invention comprises at least the following steps: c) an anion exchange chromatography and/or gel permeation chromatography treatment of the biological sample, and d) a filtration step, particularly sterilising filtration, of the material harvested after stage c). In a first variant, the process according to the invention comprises: c) an anion exchange chromatography treatment of the biological sample, and d) a filtration step, particularly sterilising filtration, on the material harvested after step c).

In another variant, the process according to the invention comprises: c) a gel permeation chromatography treatment of the biological sample, and d) a filtration step, particularly sterilising filtration, on the material harvested after step c). According to a third variant, the process according to the invention comprises: c) an anionic exchange treatment of the biological sample followed or preceded by gel permeation chromatography, and d) a filtration step, particularly sterilising filtration, on the material harvested after step c).

Embodiments of the disclosure include methods of inhibition, and/or treating Amyotrophic Lateral Sclerosis (ALS) comprising administration of a population of fibroblasts capable of inducing a regenerative and/or immunomodulatory effect in a patient suffering from ALS. In some embodiments, the fibroblasts are allogeneic to the recipient, and in some embodiments, the fibroblasts are either autologous or xenogeneic to the recipient. In certain cases, the fibroblasts are mitotically active prior to administration into a recipient in need of treatment. The fibroblasts may be isolated from a tissue selected from a group comprising of: a) skin; b) bone marrow; c) blood; d) mobilized peripheral blood; e) gingiva; f) tonsil; g) placenta; h) Wharton's Jelly; i) hair follicle; j) fallopian tube; k) liver; l) deciduous tooth; m) vas deferens; n) endometrial; o) menstrual blood; and p) omentum. The ALS may be associated with an elevation of one or more inflammatory cytokines as compared to an age-matched healthy control, such as an elevation of IL-1, IL-2, IL-6, IL-9, IL-11, IL-12, IL-15, IL-16, IL-17, IL-18, IL-20, IL-21, IL-22, IL-23, IL-27, IL-33, HMGB-1, TNF-alpha, TNF-beta, IFN-alpha, IFN-beta, and/or IFN-gamma. The fibroblasts may be administered together with a concentration of interleukin-2 sufficient to selectively upregulate activity and/or number of T regulatory cells. The interleukin-2 may be administered in the absence of fibroblasts. Any administration, including with interleukin-2, may also include rapamycin, N-acetylcysteine, and/or antibodies to CD3, including that are capable of enhancing proliferation and/or activity of T regulatory cells.

Any fibroblasts employed herein may comprise expression of CD73, CD70, CD105, CD16, CD55, CD37, interleukin-10 receptor, and/or interferon gamma receptor. The fibroblasts may comprise expression of CD73, and may be subsequently treated with interferon gamma, allowed to multiply for at least one cell division and subsequently administered. Any fibroblasts and/or modified fibroblasts and/or fibroblast exosomes may be administered in a manner capable of stimulating generation of T regulatory cells. The T regulatory cells express FoxP3, may comprise membrane bound TGF-beta, may suppress the ability of T cells to proliferate in response to a mitogen, and/or may suppress ability of immature dendritic cells to mature into differentiated dendritic cells. The dendritic cell maturation may be associated with upregulation of expression of markers selected from the group consisting of: a) HLA-II; b) CD40; c) CD80; and/or d) CD86. The dendritic cell maturation may be associated with enhanced ability to activate proliferation of allogeneic T cells and/or enhanced ability to induce production of interferon gamma from allogeneic T cells. The T regulatory cells may be activated by exposure to CD3 and/or CD28 and/or IL-10 and/or the T regulatory cells may be activated by administration of immature dendritic cells. The immature dendritic cells may express PD-1L, may be kept in an immature state by culture in low dose GM-CSF, may be kept in an immature state by culture in human chorionic gonadotropin, may be kept in an immature state by culture in hypoxia, and/or may be kept in an immature state by inhibition of NF-kappa b activity. Inhibition of NF-kappa B activity may be achieved by administration of an antisense molecule targeting NF-kappa B or molecules in the NF-kappa B pathway, by administration of a molecule capable of triggering RNA interference targeting NF-kappa B or molecules in the NF-kappa B pathway, by gene editing means targeting NF-kappa B or molecules in the NF-kappa B pathway, and/or by administration of decoy oligonucleotides capable of blocking NF-kappa B or molecules in the NF-kappa B pathway. The small molecule blocker of NF-kappa B activity may be selected from the group consisting of: Calagualine (fern derivative), Conophylline (Ervatamia microphylla), Evodiamine (Evodiae fructus component), Geldanamycin, Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, Saline (low Na+ istonic)

The T regulatory cells may be activated by incubation with mesenchymal stem cell exosomes. The T regulatory cells may be generated in vivo by exposure of T cells to an activator of interleukin-2 receptor is capable of inducing proliferation and/or activation of CD4 CD25 T cells.

In some embodiments, the interleukin-2 receptor is activated by administration of IL-2, including aldesleukin. In specific embodiments, the IL-2, including aldesleukin, is administered every day at concentrations of 0.3×106 to 3.0×106 IU IL-2 per square meter of body surface area for 1-16 weeks

In certain embodiments, one or more immune modulatory compounds are co-administered in order to enhance generation of T regulatory cells in vivo, such as oxytocin, prolactin, IL-10, and/or IL-35.

### EXAMPLES

The following example is included to demonstrate certain non-limiting aspects of the disclosure. It should be appreciated by those of skill in the art that the techniques disclosed in the examples that follow represent techniques discovered by the inventors to function well in the practice of the disclosed subject matter. However, those of skill in the art should, in light of the present disclosure, appreciate that many changes can be made in the specific embodiments that are disclosed and still obtain a like or similar result without departing from the spirit and scope of the disclosed subject matter.

### Example 1

An individual that is at risk for having ALS, such as greater than the average person in a population, or that has ALS may be subjected to methods and compositions of the disclosure. The individual may or may not be genetically predisposed for ALS. The individual may have a relative that has or had ALS. The individual may be subjected to one or more tests to determine that they have ALS or that they are at risk for having ALS, including by genetic testing and or other analyses.

The individual may be administered a therapeutically effective amount of fibroblasts, modified fibroblasts, and/or fibroblast exosomes, and in specific embodiments a therapeutically effective amount of IL-2 is administered as well. The amount of IL-2 is sufficient to result in stimulation of T regulatory cells in the individual. The fibroblasts, modified fibroblasts, and/or fibroblast exosomes may be administered at the same time as the IL-2, prior to, and/or subsequent to IL-2 administration. One or multiple administrations to the individual may occur over a defined period, or one or multiple administrations to the individual may occur through the lifetime of the individual once initiated.

In particular embodiments, following administration of the therapy, T regulatory cells in the individual are stimulated and may express FoxP3 and/or comprise membrane bound TGF-beta. The T regulatory cells may suppress the ability of T cells to proliferate in response to a mitogen. The T regulatory cells may suppress the ability of immature dendritic cells to mature into differentiated dendritic cells, and the dendritic cell maturation may be associated with upregulation of expression of one or more markers selected from the group consisting of: a) HLA-II; b) CD40; c) CD80; d) CD86; and e) a combination thereof.

### REFERENCES

1. Peters, O. M., M. Ghasemi, and R. H. Brown, Jr., Emerging mechanisms of molecular pathology in ALS. J Clin Invest, 2015.125(5): p. 1767-79.
2. Ling, S. C., M. Polymenidou, and D. W. Cleveland, Converging mechanisms in ALS and FTD: disrupted RNA and protein homeostasis. Neuron, 2013. 79(3): p. 416-38.
3. O'Toole, O., et al., Epidemiology and clinical features of amyotrophic lateral sclerosis in Ireland between 1995 and 2004. J Neurol Neurosurg Psychiatry, 2008. 79(1): p. 30-2.
4. Armon, C., Sports and trauma in amyotrophic lateral sclerosis revisited. J Neurol Sci, 2007. 262(1-2): p. 45-53.
5. Alonso, A., et al., Incidence and lifetime risk of motor neuron disease in the United Kingdom: a population-based study. Eur J Neurol, 2009. 16(6): p. 745-51.
6. Sreedharan, J. and R. H. Brown, Jr., Amyotrophic lateral sclerosis: Problems and prospects. Ann Neurol, 2013. 74(3): p. 309-16.
7. Renton, A. E., et al., A hexanucleotide repeat expansion in C9ORF72 is the cause of chromosome 9p21-linked ALS-FTD. Neuron, 2011. 72(2): p. 257-68.
8. DeJesus-Hernandez, M., et al., Expanded GGGGCC hexanucleotide repeat in noncoding region of C9ORF72 causes chromosome 9p-linked FTD and ALS. Neuron, 2011. 72(2): p. 245-56.
9. Bonda, C., et al., Amyotrophic Lateral Sclerosis with Frontotemporal Dementia in the Presence of C9orf72 Repeat Expansion-A Case Report. Innov Clin Neurosci, 2016. 13(1-2): p. 37-9.
10. Donnelly, C. J., et al., RNA toxicity from the ALS/FTD C9ORF72 expansion is mitigated by antisense intervention. Neuron, 2013. 80(2): p. 415-28.
11. Shu, L., et al., The Association between C9orf72 Repeats and Risk of Alzheimer's Disease and Amyotrophic Lateral Sclerosis: A Meta-Analysis. Parkinsons Dis, 2016. 2016: p. 5731734.
12. Cacace, R., et al., C9orf72 G4C2 repeat expansions in Alzheimer's disease and mild cognitive impairment. Neurobiol Aging, 2013. 34(6): p. 1712 el-7.
13. Lesage, S., et al., C9orf72 repeat expansions are a rare genetic cause of parkinsonism. Brain, 2013. 136 (Pt 2): p. 385-91.
14. Lindquist, S. G., et al., Corticobasal and ataxia syndromes widen the spectrum of C9ORF72 hexanucleotide expansion disease. Clin Genet, 2013. 83(3): p. 279-83.
15. Zhao, T. X., et al., Low-dose interleukin-2 inpatients with stable ischaemic heart disease and acute coronary syndromes (LILACS): protocol and study rationale for a randomised, double-blind, placebo-controlled, phase I/II clinical trial. BMJ Open, 2018. 8(9): p. e022452.
16. Mazzini, L., et al., Stem cell therapy in amyotrophic lateral sclerosis: a methodological approach in humans. Amyotroph Lateral Scler Other Motor Neuron Disord, 2003. 4(3): p. 158-61.
17. Pittenger, M. F., et al., Multilineage potential of adult human mesenchymal stem cells. Science, 1999. 284(5411): p. 143-7.
18. Mazzini, L., et al., Autologous mesenchymal stem cells: clinical applications in amyotrophic lateral sclerosis. Neurol Res, 2006. 28(5): p. 523-6.
19. Mazzini, L., et al., Mesenchymal stem cell transplantation in amyotrophic lateral sclerosis: A Phase I clinical trial. Exp Neurol, 2010. 223(1): p. 229-37.
20. Mazzini, L., et al., Mesenchymal stromal cell transplantation in amyotrophic lateral sclerosis: a long-term safety study. Cytotherapy, 2012. 14(1): p. 56-60.
21. Oh, K. W., et al., Phase I trial of repeated intrathecal autologous bone marrow-derived mesenchymal stromal cells in amyotrophic lateral sclerosis. Stem Cells Transl Med, 2015. 4(6): p. 590-7.
22. Rushkevich, Y. N., et al., The Use of Autologous Mesenchymal Stem Cells for Cell Therapy of Patients with Amyotrophic Lateral Sclerosis in Belarus. Bull Exp Biol Med, 2015. 159(4): p. 576-81.
23. Baek, W., et al., Stem cell transplantation into the intraventricular space via an Ommaya reservoir in a patient with amyotrophic lateral sclerosis. J Neurosurg Sci, 2012. 56(3): p. 261-3.
24. Sundaresan, N. and N. D. Suite, Optimal use of the Ommaya reservoir in clinical oncology. Oncology (Williston Park), 1989. 3(12): p. 15-22; discussion 23.
25. Quattrocchi, K. B., et al., Pilot study of local autologous tumor infiltrating lymphocytes for the treatment of recurrent malignant gliomas. J Neurooncol, 1999. 45(2): p. 141-57.
26. Jung, G., et al., Local immunotherapy of glioma patients with a combination of 2 bispecific antibody fragments and resting autologous lymphocytes: evidence for in situ t-cell activation and therapeutic efficacy. Int J Cancer, 2001. 91(2): p. 225-30.
27. Clemons-Miller, A. R., et al., Intrathecal cytotoxic T-cell immunotherapy for metastatic leptomeningeal melanoma. Clin Cancer Res, 2001. 7(3 Suppl): p. 917s-924s.
28. Yamanaka, R., et al., Vaccination of recurrent glioma patients with tumour lysate-pulsed dendritic cells elicits immune responses: results of a clinical phase I/II trial. Br J Cancer, 2003. 89(7): p. 1172-9.
29. Ferensztajn-Rochowiak, E. and J. K. Rybakowski, The effect of lithium on hematopoietic, mesenchymal and neural stem cells. Pharmacol Rep, 2016. 68(2): p. 224-30.
30. Yoneyama, M., et al., Lithium promotes neuronal repair and ameliorates depression-like behavior following trimethyltin-induced neuronal loss in the dentate gyrus. PLoS One, 2014. 9(2): p. e87953.
31. Boku, S., et al., Valproate recovers the inhibitory effect of dexamethasone on the prolferation of the adult dentate gyrus-derived neural precursor cells via GSK-3beta and beta-catenin pathway. Eur J Pharmacol, 2014. 723: p. 425-30.
32. Belayev, L., et al., A novel neurotrophic therapeutic strategy for experimental stroke. Brain Res, 2009. 1280: p. 117-23.
33. Boll, M. C., et al., Clinical and biological changes under treatment with lithium carbonate and valproic acid in sporadic amyotrophic lateral sclerosis. J Neurol Sci, 2014. 340(1-2): p. 103-8.
34. Jyonouchi, S., et al., Phase I trial of low-dose interleukin 2 therapy inpatients with Wiskott-Aldrich syndrome. Clin Immunol, 2017. 179: p. 47-53.
35. Asano, T., et al., Phase I/IIa Study of Low Dose Subcutaneous Interleukin-2 (IL-2) for Treatment of Refractory Chronic Graft Versus Host Disease. Acta Med Okayama, 2016. 70(5): p. 429-433.
36. Kennedy-Nasser, A. A., et al., Ultra low-dose IL-2 for GVHD prophylaxis after allogeneic hematopoietic stem cell transplantation mediates expansion of regulatory T cells without diminishing antiviral and antileukemic activity. Clin Cancer Res, 2014. 20(8): p. 2215-25.
37. Mizui, M. and G. C. Tsokos, Low-Dose IL-2 in the Treatment of Lupus. Curr Rheumatol Rep, 2016. 18(11): p. 68.
38. Todd, J. A., et al., Regulatory T Cell Responses in Participants with Type 1 Diabetes after a Single Dose of Interleukin-2: A Non-Randomised, Open Label, Adaptive Dose-Finding Trial. PLoS Med, 2016. 13(10): p. e1002139.
39. Pham, M. N., M. G. von Herrath, and J. L. Vela, Antigen-Specific Regulatory T Cells and Low Dose of IL-2 in Treatment of Type 1 Diabetes. Front Immunol, 2015. 6: p. 651.
40. Waldron-Lynch, F., et al., Rationale and study design of the Adaptive study of IL-2 dose on regulatory T cells in type 1 diabetes (DILT1D): a non-randomised, open label, adaptive dose finding trial. BMJ Open, 2014. 4(6): p. e005559.
41. von Bahr, V., et al., Mesenchymal stem cells may ameliorate inflammation in an ex vivo model of extracorporeal membrane oxygenation. Perfusion, 2019. 34(1_suppl): p. 15-21.
42. Huppert, L. A., K. D. Liu, and M. A. Matthay, Therapeutic potential of mesenchymal stromal cells in the treatment of ARDS. Transfusion, 2019. 59(S1): p. 869-875.
43. Jung, Y. J., et al., The effect of human adipose-derived stem cells on lipopolysaccharide-induced acute respiratory distress syndrome in mice. Ann Transl Med, 2019. 7(22): p. 674.
44. Chen, C. H., et al., Effective protection against acute respiratory distress syndrome sepsis injury by combined adipose-derived mesenchymal stem cells and preactivated disaggregated platelets. Oncotarget, 2017. 8(47): p. 82415-82429.
45. Lu, H., et al., Pulmonary Retention of Adipose Stromal Cells Following Intravenous Delivery Is Markedly Altered in the Presence of ARDS. Cell Transplant, 2016. 25(9): p. 1635-1643.
46. Zheng, G., et al., Treatment of acute respiratory distress syndrome with allogeneic adipose-derived mesenchymal stem cells: a randomized, placebo-controlled pilot study. Respir Res, 2014. 15: p. 39.
47. Lu, Z., et al., Mesenchymal stem cells induce dendritic cell immune tolerance via paracrine hepatocyte growth factor to alleviate acute lung injury. Stem Cell Res Ther, 2019. 10(1): p. 372.
48. Silva, J. D., et al., Mesenchymal Stromal Cells Are More Effective Than Their Extracellular Vesicles at Reducing Lung Injury Regardless of Acute Respiratory Distress Syndrome Etiology. Stem Cells Int, 2019. 2019: p. 8262849.
49. Xu, A. L., et al., Mesenchymal Stem Cells Reconditioned in Their Own Serum Exhibit Augmented Therapeutic Properties in the Setting of Acute Respiratory Distress Syndrome. Stem Cells Transl Med, 2019. 8(10): p. 1092-1106.
50. Cardenes, N., et al., Cell therapy for ARDS: efficacy of endobronchial versus intravenous administration and biodistribution of MAPCs in a large animal model. BMJ Open Respir Res, 2019. 6(1): p. e000308.
51. Li, L., et al., Mesenchymal stem cells with downregulated Hippo signaling attenuate lung injury in mice with lipopolysaccharide induced acute respiratory distress syndrome. Int J Mol Med, 2019. 43(3): p. 1241-1252.
52. Mokhber Dezfouli, M. R., et al., Intrapulmonary autologous transplant of bone marrow-derived mesenchymal stromal cells improves lipopolysaccharide-induced acute respiratory distress syndrome in rabbit. Crit Care, 2018. 22(1): p. 353.
53. Schwede, M., et al., Effects of bone marrow-derived mesenchymal stromal cells on gene expression in human alveolar type II cells exposed to TNF-alpha, IL-1beta, and IFN-gamma. Physiol Rep, 2018. 6(16): p. e13831.
54. Masterson, C., et al., Syndecan-2-positive, Bone Marrow-derived Human Mesenchymal Stromal Cells Attenuate Bacterial-induced Acute Lung Injury and Enhance Resolution of Ventilator-induced Lung Injury in Rats. Anesthesiology, 2018. 129(3): p. 502-516.
55. Park, J., et al., Expression profile of microRNAs following bone marrow-derived mesenchymal stem cell treatment in lipopolysaccharide-induced acute lung injury. Exp Ther Med, 2018. 15(6): p. 5495-5502.
56. Pedrazza, L., et al., Mesenchymal stem cells improves survival in LPS-induced acute lung injury acting through inhibition of NETs formation. J Cell Physiol, 2017. 232(12): p. 3552-3564.
57. Yang, Y., et al., The Vascular Endothelial Growth Factors-Expressing Character of Mesenchymal Stem Cells Plays a Positive Role in Treatment of Acute Lung Injury In Vivo. Mediators Inflamm, 2016. 2016: p. 2347938.
58. Moodley, Y., et al., Human mesenchymal stem cells attenuate early damage in a ventilated pig model of acute lung injury. Stem Cell Res, 2016. 17(1): p. 25-31.
59. Hayes, M., et al., Mesenchymal stromal cells are more effective than the MSC secretome in diminishing injury and enhancing recovery following ventilator-induced lung injury. Intensive Care Med Exp, 2015. 3(1): p. 29.
60. Monsel, A., et al., Therapeutic Effects of Human Mesenchymal Stem Cell-derived Microvesicles in Severe Pneumonia in Mice. Am J Respir Crit Care Med, 2015. 192(3): p. 324-36.
61. Hao, Q., et al., Study of Bone Marrow and Embryonic Stem Cell-Derived Human Mesenchymal Stem Cells for Treatment of Escherichia coli Endotoxin-Induced Acute Lung Injury in Mice. Stem Cells Transl Med, 2015. 4(7): p. 832-40.
62. Devaney, J., et al., Human mesenchymal stromal cells decrease the severity of acute lung injury induced by E. coli in the rat. Thorax, 2015. 70(7): p. 625-35.
63. Asmussen, S., et al., Human mesenchymal stem cells reduce the severity of acute lung injury in a sheep model of bacterial pneumonia. Thorax, 2014. 69(9): p. 819-25.
64. Shalaby, S. M., et al., Mesenchymal stromal cell injection protects against oxidative stress in Escherichia coli-induced acute lung injury in mice. Cytotherapy, 2014. 16(6): p. 764-75.
65. Bustos, M. L., et al., Activation of human mesenchymal stem cells impacts their therapeutic abilities in lung injury by increasing interleukin (IL)-10 and IL-IRN levels. Stem Cells Transl Med, 2013. 2(11): p. 884-95.
66. Rojas, M., et al., Infusion of freshly isolated autologous bone marrow derived mononuclear cells prevents endotoxin-induced lung injury in an ex-vivo perfused swine model. Stem Cell Res Ther, 2013. 4(2): p. 26.
67. Yan, X., et al., Nrf2 Keap1/ARE Signaling Mediated an Antioxidative Protection of Human Placental Mesenchymal Stem Cells of Fetal Origin in Alveolar Epithelial Cells. Oxid Med Cell Longev, 2019. 2019: p. 2654910.
68. Cui, P., et al., Human amnion-derived mesenchymal stem cells alleviate lung injury induced by white smoke inhalation in rats. Stem Cell Res Ther, 2018. 9(1): p. 101.
69. Zhang, S., et al., Nrf2 transfection enhances the efficacy of human amniotic mesenchymal stem cells to repair lung injury induced by lipopolysaccharide. J Cell Biochem, 2018. 119(2): p. 1627-1636.
70. Huang, Z., et al., Transcriptomic analysis of lung tissues after hUC-MSCs and FTY720 treatment of lipopolysaccharide-induced acute lung injury in mouse models. Int Immunopharmacol, 2018. 63: p. 26-34.
71. Xuan, Y. Y., et al., Human Mesenchymal Stem Stromal Cells From Human Umbilical Cord Ameliorate Acute Respiratory Distress Syndrome in Rats: Factors to Consider. Crit Care Med, 2017. 45(7): p. e736-e737.
72. Lee, F. Y., et al., Xenogeneic human umbilical cord-derived mesenchymal stem cells reduce mortality in rats with acute respiratory distress syndrome complicated by sepsis. Oncotarget, 2017. 8(28): p. 45626-45642.
73. Zhu, H., et al., Therapeutic Effects of Human Umbilical Cord-Derived Mesenchymal Stem Cells in Acute Lung Injury Mice. Sci Rep, 2017. 7: p. 39889.
74. Curley, G. F., et al., Cryopreserved, Xeno-Free Human Umbilical Cord Mesenchymal Stromal Cells Reduce Lung Injury Severity and Bacterial Burden in Rodent Escherichia coli-Induced Acute Respiratory Distress Syndrome. Crit Care Med, 2017. 45(2): p. e202-e212.
75. Chang, Y., et al., Intratracheal administration of umbilical cord blood-derived mesenchymal stem cells in a patient with acute respiratory distress syndrome. J Korean Med Sci, 2014. 29(3): p. 438-40.
76. Moodley, Y., et al., Human umbilical cord mesenchymal stem cells reduce fibrosis of bleomycin-induced lung injury. Am J Pathol, 2009. 175(1): p. 303-13.
77. Xiang, B., et al., Transplantation of Menstrual Blood-Derived Mesenchymal Stem Cells Promotes the Repair of LPS-Induced Acute Lung Injury. Int J Mol Sci, 2017. 18(4).
78. Wang, L., et al., Lung-Resident Mesenchymal Stem Cells Promote Repair of LPS-Induced Acute Lung Injury via Regulating the Balance of Regulatory T cells and Th17 cells. Inflammation, 2019. 42(1): p. 199-210.
79. Silva, J. D., et al., Mesenchymal Stem Cells From Bone Marrow, Adipose Tissue, and Lung Tissue Differentially Mitigate Lung and Distal Organ Damage in Experimental Acute Respiratory Distress Syndrome. Crit Care Med, 2018. 46(2): p. e132-e140.
80. Su, V. Y., et al., Mesenchymal Stem Cell-Conditioned Medium Induces Neutrophil Apoptosis Associated with Inhibition of the NF-kappaB Pathway in Endotoxin-Induced Acute Lung Injury. Int J Mol Sci, 2019. 20(9).
81. Mohammadipoor, A., et al., Therapeutic potential of products derived from mesenchymal stem stromal cells in pulmonary disease. Respir Res, 2018. 19(1): p. 218.
82. Lee, J. H., J. Park, and J. W. Lee, Therapeutic use of mesenchymal stem cell-derived extracellular vesicles in acute lung injury. Transfusion, 2019. 59(S1): p. 876-883.
83. Abreu, S. C., D. J. Weiss, and P. R. Rocco, Extracellular vesicles derived from mesenchymal stromal cells: a therapeutic option in respiratory diseases? Stem Cell Res Ther, 2016. 7(1): p. 53.
84. Monsel, A., et al., Mesenchymal stem cell derived secretome and extracellular vesicles for acute lung injury and other inflammatory lung diseases. Expert Opin Biol Ther, 2016. 16(7): p. 859-71.
85. Liu, F. B., Q. Lin, and Z. W. Liu, A study on the role of apoptotic human umbilical cord mesenchymal stem cells in bleomycin-induced acute lung injury in rat models. Eur Rev Med Pharmacol Sci, 2016. 20(5): p. 969-82.
86. Chen, J., et al., Mesenchymal Stem Cell Conditioned Medium Promotes Prolferation and Migration of Alveolar Epithelial Cells under Septic Conditions In Vitro via the JNK-P38 Signaling Pathway. Cell Physiol Biochem, 2015. 37(5): p. 1830-46.
87. Ionescu, L., et al., Stem cell conditioned medium improves acute lung injury in mice: in vivo evidence for stem cell paracrine action. Am J Physiol Lung Cell Mol Physiol, 2012. 303(11): p. L967-77.
88. Court, A. C., et al., Mitochondrial transfer from MSCs to T cells induces Treg differentiation and restricts inflammatory response. BMBO Rep, 2020. 21(2): p. e48052.
89. Liu, Y., et al., Human umbilical cord mesenchymal stem cells confer potent immunosuppressive effects in Sjogren's syndrome by inducing regulatory T cells. Mod Rheumatol, 2020: p. 1-11.
90. Fakhimi, M., et al., Helios, CD73 and CD39 Induction in Regulatory T Cells Exposed to Adipose Derived Mesenchymal Stem Cells. Cell J, 2020. 22(2): p. 236-244.
91. Khosravi, M., et al., Induction of CD4(+)CD25(+)FOXP3(+) regulatory T cells by mesenchymal stem cells is associated with modulation of ubiquitination factors and TSDR demethylation. Stem Cell Res Ther, 2018. 9(1): p. 273.
92. Roux, C., et al., Immunosuppressive Mesenchymal Stromal Cells Derived from Human-Induced Pluripotent Stem Cells Induce Human Regulatory T Cells In Vitro and In Vivo. Front Immunol, 2017. 8: p. 1991.
93. Miyagawa, I., et al., Regulatory Mechanism of The Induction of Regulatory T Cells through Growth Factors Released by Human Mesenchymal Stem Cells. Crit Rev Immunol, 2018. 38(6): p. 471-478.
94. Khosravi, M., et al., Induction of CD4(+)CD25(+)Foxp3(+) regulatory T cells by mesenchymal stem cells is associated with RUNX complex factors. Immunol Res, 2018. 66(1): p. 207-218.
95. Khosravi, M., et al., Mesenchymal stem cells can induce regulatory T cells via modulating miR-126a but not miR-10a. Gene, 2017. 627: p. 327-336.
96. Lee, H. J., et al., ICOSL expression in human bone marrow-derived mesenchymal stem cells promotes induction of regulatory T cells. Sci Rep, 2017. 7: p. 44486.
97. Chen, C., et al., Mesenchymal stem cells upregulate Treg cells via sHLA-G in SLE patients. Int Immunopharmacol, 2017. 44: p. 234-241.
98. Lim, J. Y., et al., Enhanced immunoregulation of mesenchymal stem cells by IL-10-producing type 1 regulatory T cells in collagen-induced arthritis. Sci Rep, 2016. 6: p. 26851.
99. Lee, E. S., et al., Adoptive Transfer of Treg Cells Combined with Mesenchymal Stem Cells Facilitates Repopulation of Endogenous Treg Cells in a Murine Acute GVHD Model. PLoS One, 2015. 10(9): p. e0138846.
100. Cahill, E. F., et al., Jagged-1 is required for the expansion of CD4+ CD25+ FoxP3+ regulatory T cells and tolerogenic dendritic cells by murine mesenchymal stromal cells. Stem Cell Res Ther, 2015. 6: p. 19.
101. Wang, Z. X., et al., Mesenchymal stem cells alleviate atherosclerosis by elevating number and function of CD4(+)CD25 (+)FOXP3 (+) regulatory T-cells and inhibiting macrophage foam cell formation. Mol Cell Biochem, 2015. 400(1-2): p. 163-72.
102. Takahashi, T., et al., Multipotent mesenchymal stromal cells synergize with costimulation blockade in the inhibition of immune responses and the induction of Foxp3+ regulatory T cells. Stem Cells Transl Med, 2014. 3(12): p. 1484-94.
103. Frazier, T. P., et al., Human adipose-derived stromal stem cells induce functional CD4+CD25+FoxP3+CD127− regulatory T cells under low oxygen culture conditions. Stem Cells Dev, 2014. 23(9): p. 968-77.
104. Li, J. G., et al., Human mesenchymal stem cells elevate CD4+CD25+CD127low/− regulatory T cells of asthmatic patients via heme oxygenase-1. Iran J Allergy Asthma Immunol, 2013. 12(3): p. 228-35.
105. Luz-Crawford, P., et al., Mesenchymal stem cells generate a CD4+CD25+Foxp3+ regulatory T cell population during the differentiation process of Th1 and Th17 cells. Stem Cell Res Ther, 2013. 4(3): p. 65.
106. Melief, S. M., et al., Multipotent stromal cells induce human regulatory T cells through a novel pathway involving skewing of monocytes toward anti-inflammatory macrophages. Stem Cells, 2013. 31(9): p. 1980-91.
107. Erkers, T., et al., Decidual stromal cells promote regulatory T cells and suppress alloreactivity in a cell contact-dependent manner. Stem Cells Dev, 2013. 22(19): p. 2596-605.
108. Engela, A. U., et al., Human adipose-tissue derived mesenchymal stem cells induce functional de-novo regulatory T cells with methylated FOXP3 gene DNA. Clin Exp Immunol, 2013. 173(2): p. 343-54.
109. Tasso, R., et al., Mesenchymal stem cells induce functionally active T-regulatory lymphocytes in a paracrine fashion and ameliorate experimental autoimmune uveitis. Invest Ophthalmol Vis Sci, 2012. 53(2): p. 786-93.
110. Choi, Y. S., J. A. Jeong, and D. S. Lim, Mesenchymal stem cell-mediated immature dendritic cells induce regulatory T cell-based immunosuppressive effect. Immunol Invest, 2012. 41(2): p. 214-29.
111. Sun, J., et al., Intrapulmonary delivery of human umbilical cord mesenchymal stem cells attenuates acute lung injury by expanding CD4+CD25+ Forkhead Boxp3 (FOXP3)+ regulatory T cells and balancing anti-and pro-inflammatory factors. Cell Physiol Biochem, 2011. 27(5): p. 587-96.
112. Madec, A. M., et al., Mesenchymal stem cells protect NOD mice from diabetes by inducing regulatory T cells. Diabetologia, 2009. 52(7): p. 1391-9.
113. English, K., et al., Cell contact, prostaglandin E(2) and transforming growth factor beta 1 play non-redundant roles in human mesenchymal stem cell induction of CD4+CD25 (High)forkhead box P3+ regulatory T cells. Clin Exp Immunol, 2009. 156(1): p. 149-60.
114. Gonzalez-Rey, E., et al., Human adipose-derived mesenchymal stem cells reduce inflammatory and T cell responses and induce regulatory T cells in vitro in rheumatoid arthritis. Ann Rheum Dis, 2010. 69(1): p. 241-8.
115. Casiraghi, F., et al., Pretransplant infusion of mesenchymal stem cells prolongs the survival of a semiallogeneic heart transplant through the generation of regulatory T cells. J Immunol, 2008. 181(6): p. 3933-46.
116. Prevosto, C., et al., Generation of CD4+ or CD8+ regulatory T cells upon mesenchymal stem cell-lymphocyte interaction. Haematologica, 2007. 92(7): p. 881-8.
117. Davis, L. S., M. C. Wacholtz, and P. E. Lipsky, The induction of T cell unresponsiveness by rapidly modulating CD3. J Immunol, 1989. 142(4): p. 1084-94.
118. Anasetti, C., et al., Induction of specific nonresponsiveness in unprimed human T cells by anti-CD3 antibody and alloantigen. J Exp Med, 1990. 172(6): p. 1691-700.
119. Woodle, E. S., et al., Phase I trial of a humanized, Fc receptor nonbinding OKT3 antibody, huOKT3gamma1 (Ala-Ala) in the treatment of acute renal allograft rejection. Transplantation, 1999. 68(5): p. 608-16.
120. Cosimi, A. B., et al., Treatment of acute renal allograft rejection with OKT3 monoclonal antibody. Transplantation, 1981. 32(6): p. 535-9.
121. Ortho Multicenter Transplant Study, G., A randomized clinical trial of OKT3 monoclonal antibody for acute rejection of cadaveric renal transplants. N Engl J Med, 1985. 313(6): p. 337-42.
122. Debure, A., et al., One-month prophylactic use of OKT3 in cadaver kidney transplant recipients. Transplantation, 1988. 45(3): p. 546-53.
123. Oh, H. K., et al., Two low-dose OKT3 induction regimens following renal transplantation—clinical experience at a single center. Clin Transplant, 1998. 12(4): p. 343-7.
124. Opelz, G., Efficacy of rejection prophylaxis with OKT3 in renal transplantation. Collaborative Transplant Study. Transplantation, 1995. 60(11): p. 1220-4.
125. Darby, C. R., et al., Reduced dose OKT3 prophylaxis in sensitised kidney recipients. Transpl Int, 1996. 9(6): p. 565-9.
126. Ciancio, G., et al., Human donor bone marrow cells can enhance hyporeactivity in renal transplantation using maintenance FK 506 and OKT3 induction therapy. Transplant Proc, 1996. 28(2): p. 943-4.
127. Waid, T. H., et al., Treatment of renal allograft rejection with T10B9. 1A31 or OKT3: final analysis of a phase II clinical trial. Transplantation, 1997. 64(2): p. 274-81.
128. Kumar, M. S., et al., ATGAM versus OKT3 induction therapy in cadaveric kidney transplantation: patient and graft survival, CD3 subset, infection, and cost analysis. Transplant Proc, 1998. 30(4): p. 1351-2.
129. Cosimi, A. B., et al., A randomized clinical trial comparing OKT3 and steroids for treatment of hepatic allograft rejection. Transplantation, 1987. 43(1): p. 91-5.
130. Millis, J. M., et al., Randomized prospective trial of OKT3for early prophylaxis of rejection after liver transplantation. Transplantation, 1989. 47(1): p. 82-8.
131. Whiting, J. F., et al., Use of low-dose OKT3 as induction therapy in liver transplantation. Transplantation, 1998. 65(4): p. 577-80.
132. Melzer, J. S., et al., The use of OKT3 in combined pancreas-kidney allotransplantation. Transplant Proc, 1990. 22(2): p. 634-5.
133. Sindhi, R., et al., Increased risk of pulmonary edema in diabetic patients undergoing preemptive pancreas transplantation with OKT3 induction. Transplant Proc, 1995. 27(6): p. 3016-7.
134. Stratta, R. J., et al., A prospective randomized trial of OKT3 vs ATGAM induction therapy in pancreas transplant recipients. Transplant Proc, 1996. 28(2): p. 917-8.
135. Ross, D. J., et al., Delayed development of obliterative bronchiolitis syndrome with OKT3 after unilateral lung transplantation. A plea for multicenter immunosuppressive trials. Chest, 1996. 109(4): p. 870-3.
136. van Gelder, T., et al., A randomized trial comparing safety and efficacy of OKT3 and a monoclonal anti-interleukin-2 receptor antibody (BT563) in the prevention of acute rejection after heart transplantation. Transplantation, 1996. 62(1): p. 51-5.
137. Delgado, J. F., et al., Induction treatment with monoclonal antibodies for heart transplantation. Transplant Rev (Orlando), 2011. 25(1): p. 21-6.
138. Kormos, R. L., et al., Monoclonal versus polyclonal antibody therapy for prophylaxis against rejection after heart transplantation. J Heart Transplant, 1990. 9(1): p. 1-9, discussion 9-10.
139. Rabinov, M., et al., Recipient selection algorithm for immunosuppression in cardiac transplantation: OKT3 vs triple therapy alone. Transplant Proc, 1992. 24(1): p. 167-8.
140. Chin, C., et al., Induction therapy for pediatric and adult heart transplantation: comparison between OKT3 and daclizumab. Transplantation, 2005. 80(4): p. 477-81.
141. Prentice, H. G., et al., Use of anti-T-cell monoclonal antibody OKT3 to prevent acute graft-versus-host disease in allogeneic bone-marrow transplantation for acute leukaemia. Lancet, 1982. 1(8274): p. 700-3.
142. Weinshenker, B. G., et al., An open trial of OKT3 in patients with multiple sclerosis. Neurology, 1991. 41(7): p. 1047-52.
143. Herold, K. C., et al., A single course of anti-CD3 monoclonal antibody hOKT3gamma1 (Ala-Ala) results in improvement in C-peptide responses and clinical parameters for at least 2 years after onset of type 1 diabetes. Diabetes, 2005. 54(6): p. 1763-9.
144. Ferran, C., et al., Cytokine-related syndrome following injection of anti-CD3 monoclonal antibody: further evidence for transient in vivo T cell activation. Eur J Immunol, 1990. 20(3): p. 509-15.
145. Vasquez, E. M., A. J. Fabrega, and R. Pollak, OKT3-induced cytokine-release syndrome: occurrence beyond the second dose and association with rejection severity. Transplant Proc, 1995. 27(1): p. 873-4.
146. Norman, D. J., J. A. Kimball, and J. M. Barry, Cytokine-release syndrome: differences between high and low doses of OKT3. Transplant Proc, 1993. 25(2 Suppl 1): p. 35-8.
147. Goldman, M., et al., OKT3-induced cytokine release attenuation by high-dose methylprednisolone. Lancet, 1989. 2(8666): p. 802-3.
148. Fletcher, E. A. K., et al., Extracorporeal human whole blood in motion, as a tool to predict first-infusion reactions and mechanism-of-action of immunotherapeutics. Int Immunopharmacol, 2018. 54: p. 1-11.
149. Chatenoud, L., et al., In vivo cell activation following OKT3 administration. Systemic cytokine release and modulation by corticosteroids. Transplantation, 1990. 49(4): p. 697-702.
150. Chatenoud, L., et al., Corticosteroid inhibition of the OKT3-induced cytokine-related syndrome—dosage and kinetics prerequisites. Transplantation, 1991. 51(2): p. 334-8.
151. Bugelski, P. J., et al., Monoclonal antibody-induced cytokine-release syndrome. Expert Rev Clin Immunol, 2009. 5(5): p. 499-521.
152. Barel, D., et al., Enhanced tumor necrosis factor in anti-CD3 antibody stimulated diabetic NOD mice: modulation by PGE1 and dietary lipid. Autoimmunity, 1992. 13(2): p. 141-9.
153. Pradier, O., et al., Procoagulant effect of the OKT3 monoclonal antibody: involvement of tumor necrosis factor. Kidney Int, 1992. 42(5): p. 1124-9.
154. Wissing, K. M., et al., A pilot trial of recombinant human interleukin-10 in kidney transplant recipients receiving OKT3 induction therapy. Transplantation, 1997. 64(7): p. 999-1006.
155. Nakamoto, S., Reflections on My Lifetime Teacher: Dr. Willem J. Kolff. Artif Organs, 2018. 42(2): p. 115-126.
156. Krakauer, R. S., et al., Circulating immune complexes in rheumatoid arthritis. Selective removal by cryogelation with membrane filtration. Arch Intern Med, 1982. 142(2): p. 395-7.
157. Snyder, H. W., Jr., et al., Clearance offline leukemia virus from persistently infected pet cats treated by extracorporeal immunoadsorption is correlated with an enhanced antibody response to FeLV gp 70. J Immunol, 1984. 132(3): p. 1538-43.
158. Hellstrom, K. E., et al., Blocking (suppressor)factors, immune complexes, and extracorporeal immunoadsorption in tumor immunity. Contemp Top Immunobiol, 1985. 15: p. 213-38.
159. Messerschmidt, G. L., et al., Protein A immunoadsorption in the treatment of malignant disease. J Clin Oncol, 1988. 6(2): p. 203-12.
160. Cameron, D. J., C. T. Fitts, and P. R. Rajagopalan, Antigen-coated immunoadsorbents utilized for in vivo depletion of antibodies and lymphocytes with specificity for the antigen. J Surg Oncol, 1983. 23(3): p. 158-62.
161. Lazaridis, K., et al., Specific removal of autoantibodies by extracorporeal immunoadsorption ameliorates experimental autoimmune myasthenia gravis. J Neuroimmunol, 2017. 312: p. 24-30.
162. Lupinek, C., et al., Extracorporeal IgE Immunoadsorption in Allergic Asthma: Safety and Efficacy. EBioMedicine, 2017. 17: p. 119-133.
163. Fabbrini, P., et al., Light chains removal by extracorporeal techniques in acute kidney injury due to multiple myeloma: a position statement of the Onconephrology Work Group of the Italian Society of Nephrology. J Nephrol, 2016. 29(6): p. 735-746.
164. Opgenoorth, M., et al., Treatment of antibody-mediated rejection including immunoadsorption during first year after renal transplantation—Clinical results and regulation of endothelial progenitor cells. Atheroscler Suppl, 2015. 18: p. 67-73.
165. Lazaridis, K., et al., Antigen-specific apheresis of autoantibodies in myasthenia gravis. Ann N Y Acad Sci, 2012. 1275: p. 7-12.
166. Koch, B., S. Buttner, and H. Geiger, [Reducing viral load in life-threatening viral diseases using snowdrops]. Dtsch Med Wochenschr, 2016. 141(25): p. 1868-1871.
167. Koch, B., et al., Lectin Affinity Plasmapheresis for Middle East Respiratory Syndrome-Coronavirus and Marburg Virus Glycoprotein Elimination. Blood Purif, 2018. 46(2): p. 126-133.
168. Moriyama, M., et al., Removal of hepatitis C virus by G-1 beads in sera from patients with chronic hepatitis C. Intervirology, 2005. 48(2-3): p. 84-8.
169. Josephs, S. F., et al., Unleashing endogenous TNF-alpha as a cancer immunotherapeutic. J Transl Med, 2018. 16(1): p. 242.
170. Kanekura, T., et al., Granulocyte and monocyte adsorption apheresis (GCAP) for refractory skin diseases caused by activated neutrophils and psoriatic arthritis: evidence that GCAP removes Mac-1-expressing neutrophils. Ther Apher Dial, 2006. 10(3): p. 247-56.
171. Nakane, S., et al., Cytapheresis with a filter for selective removal of CD4+ T cells in experimental autoimmune encephalomyelitis. Mult Scler, 2003. 9(6): p. 579-84.
172. Nicu, C., et al., Dermal adipose tissue secretes HGF to promote human hair growth and pigmentation. J Invest Dermatol, 2021.
173. Sano, S., et al., Tissue regeneration: hair follicle as a model. J Investig Dermatol Symp Proc, 2001. 6(1): p. 43-8.
174. Otomo, S., [Hair growth effect of minoxidil]. Nihon Yakurigaku Zasshi, 2002. 119(3): p. 167-74.
175. Zhou, Y., et al., Autologous activated platelet-rich plasma in hair growth: A pilot study in male androgenetic alopecia with in vitro bioactivity investigation. J Cosmet Dermatol, 2021. 20(4): p. 1221-1230

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method of treating or preventing or reducing the risk of having Amyotrophic Lateral Sclerosis (ALS) in an individual, comprising administering to the individual a therapeutically effective amount of a population of fibroblasts, fibroblast exosomes, modified fibroblasts, IL-2, or a combination thereof.

2. The method of claim 1, further comprising administering to the individual an effective amount of rapamycin, N-acetylcysteine, anti-CD3 antibodies, or a combination thereof.

3. The method of claim 1 or 2, wherein the fibroblasts are allogeneic to the individual.

4. The method of claim 1 or 2, wherein the fibroblasts are autologous or xenogeneic to the individual.

5. The method of any one of claims 1-4, wherein said fibroblasts are mitotically active prior to administration into a recipient in need of treatment.

6. The method of any one of claims 1-5, wherein said fibroblasts are isolated from a tissue selected from the group consisting of: a) skin; b) bone marrow; c) blood; d) mobilized peripheral blood; e) gingiva; f) tonsil; g) placenta; h) Wharton's Jelly; i) hair follicle; j) fallopian tube; k) liver; l) deciduous tooth; m) vas deferens; n) endometrial; o) menstrual blood; p) omentum; and q) a combination thereof.

7. The method of any one of claims 1-6, wherein said ALS in the individual is associated with an elevation of inflammatory cytokines as compared to an age-matched healthy control.

8. The method of claim 6, wherein said inflammatory cytokine is IL-1, IL-2, IL-6, IL-9, IL-11, IL-12, IL-15, IL-16, IL-17, IL-18, IL-20, IL-21, IL-22, IL-23, IL-27, IL-33, HMGB-1, TNF-alpha, TNF-beta, IFN-alpha, IFN-beta, and/or IFN-gamma.

9. The method of any one of claims 1-8, wherein said fibroblasts are selected for expression of CD73, CD70. CD105, CD16. CD55. CD37, interleukin-10 receptor, interferon gamma receptor.

10. The method of any one of claims 1-9, wherein said fibroblasts are selected for expression of CD73, subsequently treated with interferon gamma, and allowed to multiply for at least one cell division prior to administration.

11. The method of any one of claims 1-10, wherein said fibroblasts and/or modified fibroblasts are administered in a manner capable of stimulating generation of T regulatory cells.

12. The method of claim 11, wherein said T regulatory cells express FoxP3.

13. The method of claim 11 or 12, wherein said T regulatory cells comprise membrane bound TGF-beta.

14. The method of any one of claims 11-13, wherein said T regulatory cells suppress the ability of T cells to proliferate in response to a mitogen.

15. The method of any one of claims 11-14, wherein said T regulatory cells suppress the ability of immature dendritic cells to mature into differentiated dendritic cells.

16. The method of claim 15, wherein said dendritic cell maturation is associated with upregulation of expression of one or more markers selected from the group consisting of: a) HLA-II; b) CD40; c) CD80; d) CD86; and e) a combination thereof.

17. The method of claim 15 or 16, wherein said dendritic cell maturation is associated with enhanced ability to activate proliferation of allogeneic T cells.

18. The method of any one of claims 15-17, wherein said dendritic cell maturation is associated with enhanced ability to induce production of interferon gamma from allogeneic T cells.

19. The method of any one of claims 11-18, wherein said T regulatory cells are activated by exposure to CD3 and CD28.

20. The method of any one of claims 11-19, wherein said T regulatory cells are activated by exposure to interleukin-10.

21. The method of any one of claims 11-20, wherein said T regulatory cells are activated by administration of immature dendritic cells.

22. The method of claim 21, wherein said immature dendritic cells express PD-1L.

23. The method of claim 21 or 22, wherein said immature dendritic cells are kept in an immature state by culture in low dose GM-CSF.

24. The method of any one of claims 21-23, wherein said immature dendritic cells are kept in an immature state by culture in human chorionic gonadotropin.

25. The method of claim 24, wherein said immature dendritic cells are kept in an immature state by culture in hypoxia.

26. The method of claim 24 or 25, wherein said immature dendritic cells are kept in an immature state by inhibition of NF-kappa b activity.

27. The method of claim 26, wherein said inhibition of NF-kappa B activity is achieved by administration of an antisense molecule targeting NF-kappa B or molecules in the NF-kappa B pathway.

28. The method of claim 26 or 27, wherein said inhibition of NF-kappa B activity is achieved by administration of a molecule capable of triggering RNA interference targeting NF-kappa B or molecules in the NF-kappa B pathway.

29. The method of any one of claims 26-28, wherein said inhibition of NF-kappa B activity is achieved by gene editing means targeting NF-kappa B or molecules in the NF-kappa B pathway.

30. The method of any one of claim 26-29, wherein said inhibition of NF-kappa B activity is achieved by administration of decoy oligonucleotides capable of blocking NF-kappa B or molecules in the NF-kappa B pathway.

31. The method of any one of claims 26-30, wherein said inhibition of NF-kappa B activity is achieved by administration of a small molecule blocker of NF-kappa B activity.

32. The method of claim 31, wherein said small molecule blocker of NF-kappa B activity is selected from a group comprising of: Calagualine (fern derivative), Conophylline (Ervatamia microphylla), Evodiamine (Evodiae fructus component), Geldanamycin, Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, Saline (low Na+ istonic)

33. The method of any one of claims 11-32, wherein T regulatory cells are activated by incubation with mesenchymal stem cell exosomes.

34. The method of any one of claims 11-33, wherein said T regulatory cells are generated in vivo by exposure of T cells to an activator of interleukin-2 receptor is capable of inducing proliferation and/or activation of CD4 CD25 T cells.

35. The method of any one of claims 1-34, wherein said interleukin-2 receptor is activated by administration of the IL-2.

36. The method of any one of claims 1-35, wherein said IL-2 is administered every day at concentrations of 0.3×10⁶ to 3.0×10⁶ IU IL-2 per square meter of body surface area for 1-16 weeks

37. The method of any one of claims 1-36, further comprising administering one or more immune modulatory compounds.

38. The method of claim 37, wherein said compound is oxytocin, prolactin, IL-10, IL-35, CD3 inhibitor, or a combination thereof.

39. The method of claim 38, wherein the CD3 inhibitor is an anti-CD3 antibody.

40. The method of claim 39, wherein said anti-CD3 antibody is Teplizumab.

41. The method of any one of claims 1-40, wherein the individual has a familial form of ALS.

42. The method of any one of claims 1-40, wherein the individual has an idiopathic form of ALS.

43. The method of any one of claims 1-42, wherein the individual has one or more mutations in the C9ORF72 gene.

44. The method of any one of claims 1-43, further comprising administering riluzole to the individual.
